(19) United States
(12) Patent Application Publication
(10) Pub. No.: US 2005/0042293 A1
(43) Pub. Date: Feb. 24, 2005

(54) Polymeric systems for drug delivery and uses thereof

(75) Inventors: John Jackson, Vancouver (CA); Xichen Zhang, Castro Valley, CA (US); Helen Burt, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignees: The University of British Columbia, Vancouver (CA); Angiotech Pharmaceuticals, Inc., Vancouver (CA)

(21) Appl. No.: 10/721,835

(22) Filed: Nov. 25, 2003

Related U.S. Application Data
(63) Continuation of application No. 09/181,582, filed on Oct. 28, 1998, now abandoned.
(60) Provisional application No. 60/063,721, filed on Oct. 29, 1997.
(60) Provisional application No. 60/076,842, filed on Mar. 4, 1998.

(51) Int. Cl. A61K 9/14

(52) U.S. Cl. 424/486

(57) ABSTRACT

Biodegradable polymeric implants can provide a safe and efficient means to deliver drugs in the treatment of various diseases. Although a polymeric drug delivery system can be implanted as a solid device within a subject, it is also possible to administer such a system as an injectable liquid which solidifies in vivo. An improved formulation of a polymeric drug delivery system comprises a water insoluble copolymer that is a solid or wax at 37° C., a water soluble polymer that is a liquid at 25° C., and a hydrophobic drug. These drug delivery systems can be administered by injection, and do not require the use of a toxic curing agent or inconvenient temperature manipulations.

### CROSS-REFERENCE TO RELATED APPLICATIONS

The present application is a Continuation of co-pending U.S. patent application Ser. No. 09/181,582, filed Oct. 28, 1998, which claims the priority benefit of U.S. Provisional Patent Application No. 60/063,721, filed Oct. 29, 1997, and U.S. Provisional Patent Application No. 60/076,842 filed Mar. 4, 1998, where these patent applications are fully incorporated herein by reference.

### TECHNICAL FIELD

The present invention relates generally to therapeutic and prophylatic compositions and methods, and more particularly to polymeric compounds and compositions incorporating same, that may be used for the controlled release of a drug.

### BACKGROUND OF THE INVENTION

There is considerable interest in the use of controlled drug release systems for long-term treatment of various diseases. In one general approach to drug delivery, the drug-loaded systems include bioerodible polymeric materials. This type of drug delivery system is designed to provide a controlled release of a drug as the system gradually degrades within the body to non-toxic components. Such an erodible drug delivery system does not require surgical removal of the remains of an implant after use. Biodegradable drug delivery systems are described, for example, by Dunn et al. in U.S. Pat. Nos. 4,938,763; 5,278,201; 5,278,202; and 5,340,849.

Although a polymeric drug delivery system can be implanted as a solid device (composition, system) within a subject, it is also possible to administer such a system as an injectable liquid which solidifies in vivo. These systems have the advantages of a solid implant, while avoiding the need for surgery to administer the implant. One approach to designing an injectable polymeric drug delivery system is to combine a drug-polymeric solution with a solvent. When this “thermoplastic” polymeric solution is injected into a subject, the solvent diffuses away from the drug-polymeric mixture and water diffuses into the mixture, causing the solidification of the drug-polymer mixture and the formation of a solid drug implant. Typical solvents used to form thermoplastic implants include N-methyl-2-pyrrolidone, methyl ethyl ketone, dimethylformamide, and dimethylsulfoxide. A disadvantage of these thermoplastic systems is that the solvents used therein can be toxic or irritating to body tissues.

Another type of injectable polymeric drug delivery system is a thermosetting device. Here, cross-linkable polymers are cured in vivo using a curing agent which is added to the polymers immediately prior to injection. Drawbacks of such thermosetting systems include the need for rapid administration of the polymeric solution as it is curing, and the potential toxicity of the curing agent.

Other types of thermosetting systems use temperature, rather than a curing agent, for the solidification process. Davis and Scott, U.S. Pat. No. 5,384,333, for example, describe a polymeric paste that can be injected as a molten liquid that solidifies at body temperature. On the other hand, Cha et al., U.S. Pat. No. 5,702,717, describe a polymeric drug delivery system having reverse thermal gelation properties, which solidifies at body temperature. Drug delivery systems that depend on maintaining a certain injection temperature are problematic, however, because injection needles rapidly equilibrate at 37° C. once inserted into a body. Consequently, the drug delivery systems of Cha et al. have a tendency to solidify in the needle during administration.

The development of compositions for drug delivery is a very active area of current research worldwide. Drug delivery compositions which may be tolerated by the host and can be injected into a host are particularly needed. Thus, a need exists for a biodegradable polymeric drug delivery system that provides a controlled release of drug, is non-toxic, and is simple to administer in a reproducible manner. The present invention is directed to fulfilling this need, and provides further related advantages as described more fully herein.

### SUMMARY OF THE INVENTION

The present invention provides polymers, compositions containing polymers and drugs, methods of preparing the polymers and compositions, and methods of using the polymers and compositions in a therapeutically effective manner.

In one aspect, the invention provides a polymeric drug delivery system. The system includes (a) a biodegradable water insoluble polymer that is a solid or wax at 37° C.; (b) a biodegradable water soluble polymer that is a liquid at 25° C.; and (c) a hydrophobic drug, wherein the polymeric drug delivery system is a liquid or paste at 25° C.

In another aspect, the invention provides a method for delivering a drug to a subject. The method includes administrating a polymeric drug delivery system to the subject, where the polymeric drug delivery system includes (a) a biodegradable water insoluble polymer that is a solid or wax at 37° C., (b) a biodegradable water soluble polymer that is a liquid at 25° C., and (c) a hydrophobic drug, wherein said polymeric drug delivery system is a liquid or paste at 25° C.

In another aspect, the invention provides a method of preparing a polymeric drug delivery system. The method includes the blending together of: (a) a biodegradable water insoluble polymer that is a solid or wax at 37° C., (b) a biodegradable water soluble polymer that is a liquid at 25° C., and (c) a hydrophobic drug, wherein the polymeric drug delivery system is a liquid or paste at 25° C.

In another aspect, the invention provides a triblock copolymer of the formula ABA, wherein A is a block of residues that includes the residues which remain after polymerization of one or more monomers selected from hydroxyacetic acid, 2-hydroxypropionic acid and 6-hydroxyhexanoic acid, B is a block of residues that includes the residues which remain after the polymerization of one or more monomers selected from alkylene oxide and alkylene glycol, where the copolymer is either a paste or liquid at a temperature within the range of 25-40° C., or has a non-solid consistency at 25° C.

In another aspect, the invention provides a drug delivery system which includes a drug in combination with a triblock copolymer of the formula ABA, wherein A is a block of residues that includes the residues which remain after polymerization of one or more of hydroxyacetic acid, 2-hydroxypropionic acid and 6-hydroxyhexanoic acid, B is a block of residues that include residues which remain after the polymerization of one or more of alkylene oxide and alkylene glycol, and the copolymer is either a paste or a liquid at a temperature within the range of 25-40° C., or has a non-solid consistency at 25° C.

In another aspect, the invention provides a method of administering a drug to a subject wherein the subject is effectively contacted with an effective drug delivery system which includes drug in combination with a triblock copolymer of the formula ABA, wherein A is a block of residues that includes residues that result from the polymerization of one or more monomers selected from hydroxyacetic acid, 2-hydroxypropionic acid and 6-hydroxyhexanoic acid, B is a block of residues that includes residues that result from the polymerization of one or more monomers selected from alkylene oxide and alkylene glycol, and the copolymer has either a paste or liquid consistency at a temperature within the range of 25-40° C., or has a non-solid consistency at 25° C.

These and other aspects of the present invention will become evident upon reference to the following detailed description and attached drawings. In addition, various references are identified below and are incorporated by reference in their entirety.

### BRIEF DESCRIPTION OF THE DRAWINGS

FIGS. 1A and 1B show DSC thermograms of PLC-PEG-PLC 4000-35/35/30 with various paclitaxel loadings. FIG. 1A is before γ-ray irradiation; FIG. 1B is after 2.5 Mrad γ-ray irradiation.

FIGS. 2A and 2B are graphs representing the release of paclitaxel from non-irradiated (FIG. 2A) and 2.5 Mrad γ-ray irradiated (FIG. 2B) PLC-PEG-PLC 4000-35/35/30 pastes into PBSA at 37° C.

FIG. 3 is a graph representing the release of paclitaxel from PLC-PEG-PLC 2000-35/35/30 paints into PBSA at 37° C.

FIG. 4 is a graph representing the degradation of PLC-PEG-PLC 4000-35/35/30 pastes with or without paclitaxel loaded in PBSA at 37° C.

FIG. 5A is a scan showing endothermic peaks of compositions having different polymeric ratios of TB:MePEG, as analyzed by differential scanning calorimetry. The melting points of the compositions are also provided in FIG. 5A.

FIG. 5B shows the average peak melting point of various paste formulations and the linear relationship between melting point depression and an increasing proportion of MePEG. The heating rate was 10° C./min.

FIG. 5C shows DSC thermograms of TB, MePEG and combination blends of paste. The heating rate was 40° C./min. Each sample was quenched by heating it to 80° C. followed by a rapid cooling at 500° C./min. The thermograms were observed at a heating rate of 40° C./min. Samples were held in a crimped aluminum pan with an aluminum lid.

FIG. 6 is a chart comparing the time required for control and drug-loaded paste pellets to break up under stirring at 300 rpm at 37° C.

FIG. 7 is a photograph of a gel showing fragmentation of LNCaP DNA, as described in Example 6. Lane 1 (the left lane): untreated LNCaP cells; Lane 2: LNCaP cells incubated with 0.01 nM paclitaxel; Lane 3: LNCaP cells incubated with 0.1 nM paclitaxel; Lane 4: LNCaP cells incubated with 1 nM paclitaxel; Lane 5: LNCaP cells incubated with 10 nM paclitaxel; Lane 6: LNCaP cells incubated with 100 nM paclitaxel; Lane 7: DNA ladder markers.

FIGS. 8A and 8B are graphs showing drug release profiles for 10% drug-loaded TB:MePEG350 (40:60) pastes.

FIGS. 9A, 9B, 9C, and 9D are graphs showing release profiles for 2.5% (FIG. 9A), 5% (FIG. 9B), 10% (FIG. 9C), and 15% (FIG. 9D) paclitaxel-loaded pastes (15 mg) composed of TB:MePEG350 blends in the range of 30:70 to 90:10.

FIGS. 10A and 10B are graphs illustrating the disintegration of paclitaxel-loaded (2.5%, 5%, and 10% paclitaxel w/w) pastes using 30:70 to 90:10 TB:MePEG350 blends. The data are presented as the total number of paste fragments in PBS/Albumin on either day 7 or 30 (37° C.).

FIG. 11A is a graph that shows the average percentage of MePEG lost into distilled water, at a temperature 37° C. and shaken at 90 rotations per minutes (rpms), from 300 mg of the various paste formulations (no paclitaxel present). The error bars represent the standard deviation of 3 samples from the same batch.

FIG. 11B is a graph that shows the average percentage of MePEG lost into distilled water, at a temperature 37° C. and shaken at 90 rpms, from 300 mg of the various 10% paclitaxel loaded paste formulations. The error bars represent the standard deviation of 3 samples from the same batch.

FIG. 11C shows a chromatogram of GPC scans of 30:70 paste blend after incubation. The peak to the left represents TB while the peak to the right represents MePEG. Scans were performed using 20 μL injections of 0.25% polymer solution in chloroform using a chloroform mobile phase at a rate of 1 mL/min. Calculations were done using the values for the area under the curve of GPC chromatographs. Chromatogram values were validated using polystyrene standards.

FIG. 11D is a graph that shows the proportion of MePEG that remained in the paste formulations after incubation. Scans were performed using 20 μl injections of 0.25% polymer solution in chloroform using a chloroform mobile phase at a rate of 1 mL/min. Calculations were done using the values for the area under the curve of GPC chromatographs. Chromatogram values were validated using polystyrene standards.

FIGS. 12A, 12B, and 12C are graphs showing the assessment of serum prostate-specific antigen (PSA) levels in mice in the Control Group (FIG. 12A), Early Treatment Group (FIG. 12B), and Late Treatment Group (FIG. 12C). Early and Late Treatment Groups received 10% paclitaxel-loaded paste by intratumoral injection of 100 μL of paste into each tumor.

FIGS. 13A, 13B, and 13C are graphs illustrating the assessment of tumor volume in mice treated with (FIG. 13A) control, no treatment (FIG. 13B) 10% paclitaxel-loaded paste (100 μL) intratumorally at week 3 (Early Treatment Group) (FIG. 13C) 10% paclitaxel-loaded paste (100 μL) at week 5 (Late Treatment Group).

### DETAILED DESCRIPTION OF THE INVENTION

1. Definitions

In the description that follows, a number of terms are used extensively. The following definitions are provided to facilitate understanding of the invention.

As used herein, a “triblock copolymer” has three distinct blocks, preferably of alternating hydrophilic and hydrophobic blocks, where a preferred triblock copolymer is water insoluble. An exemplary preferred triblock copolymer has an ABA-type structure, such as [polyester]-[polyalkylene oxide]-[polyester], where polyester is hydrophobic and polyalkylene oxide is hydrophilic. Either of the A or B blocks may, themselves, be a copolymer.

A “blend” is a mixture of two or more components characterized by the lack of, or substantial lack of, covalent bonding between the components.

As used herein, a “polymeric blend” is a mixture of two biodegradable, biocompatible polymers, in which one polymer is water insoluble and the other polymer is water soluble. An example of a polymeric blend is a mixture of a water insoluble triblock copolymer and a water soluble polyalkylene oxide.

A “drug” is a therapeutically active substance which is delivered to a living subject to produce a desired effect, such as to treat a condition of the subject. A drug is also provided to a subject prophylactically to prevent the development of a condition or to decrease the severity of a condition that the subject may develop.

As used herein, a “hydrophobic drug,” is a water insoluble drug. A “water insoluble drug” has a solubility of less than 0.1 mg/mL in distilled water at 25° C. Within the context of the present invention, a “slightly soluble drug” (solubility: 1-10 mg/mL) and a “very slightly soluble drug” (solubility: 0.1-1 mg/mL) may also be referred to. These terms are well-known to those of skill in the art. See, e.g., Martin (ed.), Physical Pharmacy, Fourth Edition, page 213 (Lea and Febiger 1993).

As used herein, “a polymeric drug delivery system,” is a blend having a hydrophobic drug dissolved or suspended within one or more polymers.

The term “slow release” refers to the release of a drug from a polymeric drug delivery system over a period of time that is more than one day.

As used herein, the following terms are given the indicated abbreviations: poly(ε-caprolactone) (PCL); polyesters (PE); polyethylene glycol (PEG); polyglycolide (PGA); polylactide (PLA); poly(lactide-co-glycolide) (PLGA); and poly(DL-lactide-co-ε-caprolactone) (PLC).

2. Methods of Making Polymeric Drug Delivery Systems

In one aspect, the present invention provides a polymeric drug delivery system that includes a drug and a blend of a water insoluble biodegradable, biocompatible polymer and a water soluble biodegradable, biocompatible polymer. Preferably, the insoluble polymer is a triblock copolymer that is a solid or wax at body temperature (about 37° C., at normal atmospheric pressure), and has a melting point slightly greater than body temperature. As used herein, the term “wax” refers to a composition that is readily molded by application of pressure.

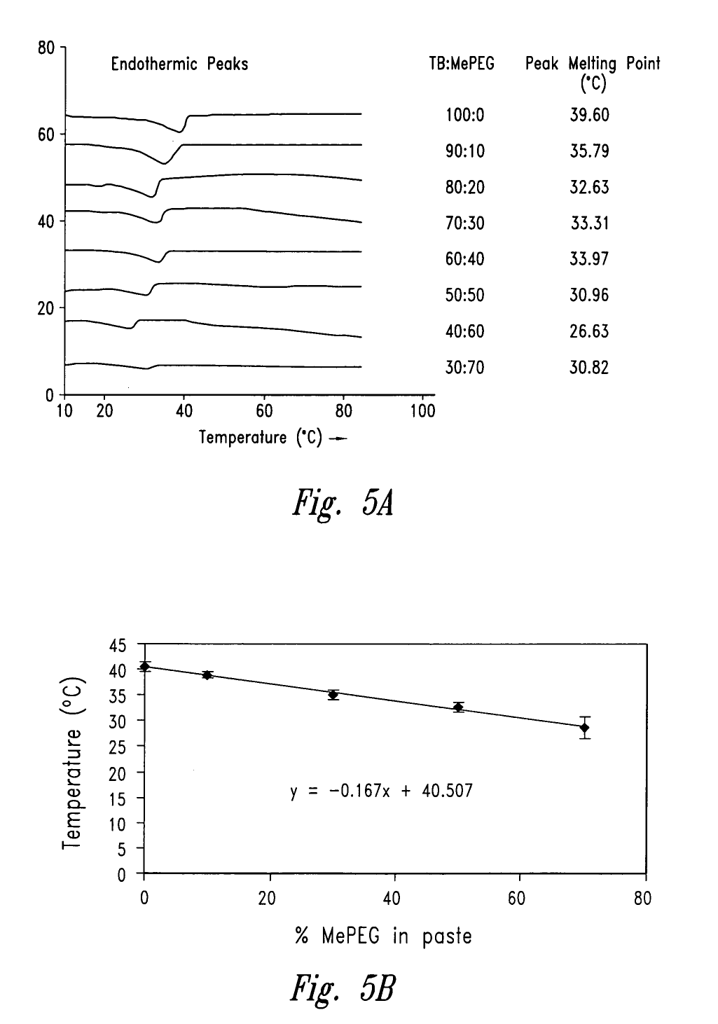

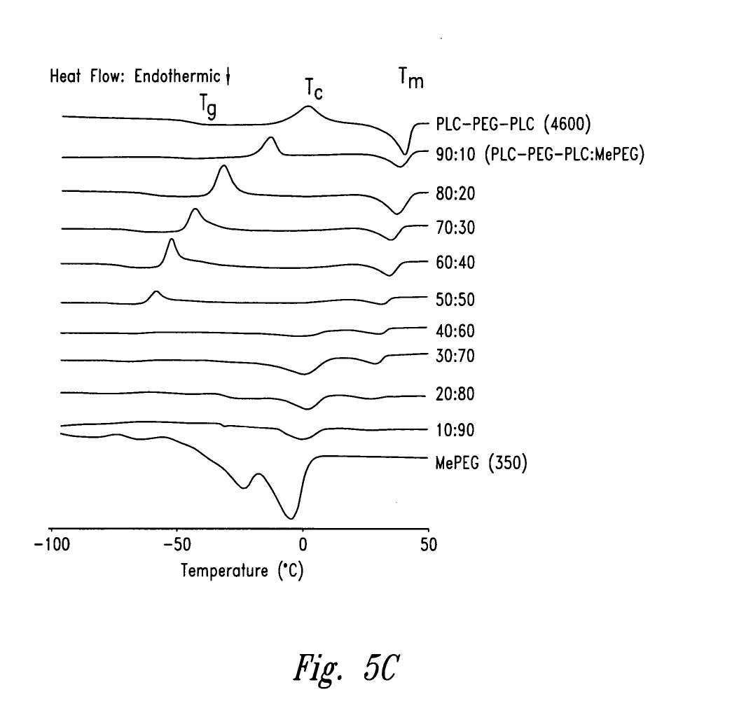

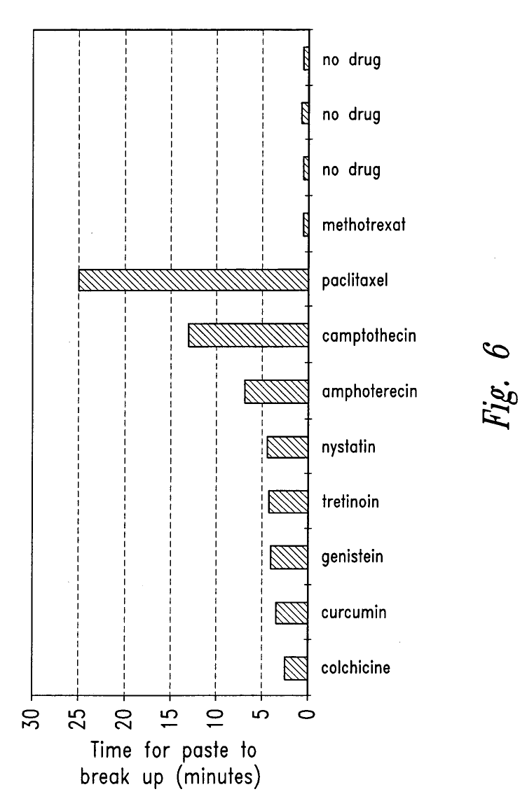

Because polymers generally are manufactured with a molecular weight range, the melting point of most polymers falls over a temperature range, rather than at a discrete temperature. This is illustrated in the top line of FIG. 5A. Over a range of temperatures, therefore, the polymer changes from a solid to a liquid, and within this range, the polymer has a waxy feel. The water insoluble polymer of the present invention should not be a liquid at 37° C., but should be a waxy solid. The blending of the second liquid polymer to such a waxy solid will therefore make the blend more fluid and enable injection through a syringe/needle assembly. Typically, the peak of the melting point range of the water insoluble polymer should lie in the 37-50° C. temperature range.

A preferred triblock copolymer of the present invention is an ABA triblock copolymer in which the A block is hydrophilic and the B block is hydrophobic. A preferred ABA triblock copolymer may be represented by the general structure [polyester]-[polyalkylene oxide]-[polyester].

Preferably, the polyester is a poly(α-hydroxy acid), such as poly(glycolic acid) or poly(lactic acid), which is hydrolyzed in vivo to its constituent α-hydroxy acids and excreted. Suitable polyalkylene oxides include polyethylene glycol (PEG) and methylated versions thereof (MPEGs). In one embodiment, for example, the ABA triblock copolymer comprises poly(lactic acid) as the A block and polyethylene glycol as the B block. Preferably, the A and B blocks of such a copolymer are bonded to each other via caprolactone links. An advantage of incorporating caprolactone links is that the resultant triblock copolymer has a fast rate of degradation in vivo. One preferred triblock copolymer of this type can be represented by the structrure [poly(DL-lactide-co-ε-caprolactone)]-[polyethylene glycol]-[poly(DL-lactide-co-ε-caprolactone)].

In another aspect, the present invention provides a triblock copolymer of the formula ABA, wherein A is a block including residues having the structure resulting from the polymerization of monomers selected from hydroxyacetic acid, 2-hydroxypropionic acid and 6-hydroxyhexanoic acid, B is a block including residues having the structure resulting from the polymerization of alkylene oxide, and the copolymer is a liquid at a temperature within the range of 25-40° C. Preferably, the A block is hydrophobic, the B block is hydrophilic, and the triblock copolymer is water-insoluble.

In another aspect, the invention provides a triblock copolymer of the formula ABA, wherein A is a block including residues having the structure resulting from the polymerization of monomers selected from hydroxyacetic acid, 2-hydroxypropionic acid and 6-hydroxyhexanoic acid, B is a block including residues having the structure resulting from the polymerization of alkylene oxide, and the copolymer is a paste at a temperature within the range of 25-40° C. Preferably, the A block is hydrophobic, the B block is hydrophilic, and the triblock copolymer is water-insoluble.

In another aspect, the invention provides a triblock copolymer of the formula ABA, wherein A is a block including residues having the structure resulting from the polymerization of monomers selected from hydroxyacetic acid, 2-hydroxypropionic acid and 6-hydroxyhexanoic acid, B is a block including residues having the structure resulting from the polymerization of alkylene oxide, and the copolymer is not a solid at 25° C. Preferably, the A block is hydrophobic, the B block is hydrophilic, and the triblock copolymer is water-insoluble.

General methods for making ABA triblock copolymers are provided, for example, by Kimura et al., Polymer 30:1342, 1989. Methods for synthesizing triblock copolymers comprising poly(ε-caprolactone) and polyethylene glycol are described, for example, by Martini et al., J. Chem. Soc. Faraday Trans. 90:1961, 1994. Moreover, methods for diblock polymer synthesis are described, for example, by Zhang et al., Anticancer Drugs 8:696 (1997), and by Ramaswamy et al., J. Pharm. Sci. 86:460 (1997).

In preferred embodiments of the invention, the ABA copolymer includes an A block that consists essentially of residues having the structure resulting from the polymerization of monomers selected from the group hydroxyacetic acid, 2-hydroxypropionic acid and 6-hydroxyhexanoic acid. In another embodiment, the A block includes residues having the structure resulting from the polymerization of 2-hydroxypropionic acid. In another embodiment, the A block consists essentially of residues having the structure resulting from the polymerization of 2-hydroxypropionic acid. In another embodiment, the A block includes residues having the structure resulting from the polymerization of 6-hydroxyhexanoic acid. In another embodiment, the A block, includes residues having the structure resulting from the polymerization of 2-hydroxypropionic acid and 6-hydroxyhexanoic acid. In another embodiment, the A block consists essentially of residues having the structure resulting from the polymerization of 2-hydroxypropionic acid and 6-hydroxyhexanoic acid. The A block may be a copolymer. In one embodiment, the A block is a block copolymer. In another embodiment, the A block is a random copolymer.

In another embodiment, the A block contains residues having the structure resulting from the polymerization of 2-hydroxypropionic acid and 6-hydroxyhexanoic acid in a 2-hydroxypropionic acid:6-hydroxyhexanoic acid weight ratio of 40-60:60-40. In a preferred embodiment, the weight ratio is 45-55:55-45.

In another embodiment, the B block includes residues having the structure resulting from the polymerization of ethylene oxide. In another embodiment, the B block consists essentially of residues having the structure resulting from the polymerization of ethylene oxide. In another embodiment, the B block includes residues having the structure resulting from the polymerization of ethylene oxide and propylene oxide. In another embodiment, the B block consists essentially of residues having the structure resulting from the polymerization of ethylene oxide and propylene oxide.

In another embodiment, the B block is a CDC triblock copolymer wherein C and D are selected from homopolymers of ethylene oxide and propylene oxide. The C block may be a homopolymer of ethylene oxide and the D block may be a homopolymer of propylene oxide. The C block may be a homopolymer of propylene oxide and the D block may be a homopolymer of ethylene oxide.

Typically, the B block has a number average molecular weight of less than or equal to 8,000. In various embodiments, the B block has a number average molecular weight of less than or equal to 5,000; less than or equal to 4,000; less than or equal to 3,000; less than or equal to 2,000; or less than or equal to 1,000. Typically, the copolymer has a number average molecular weight of the B block of at least 100, and more typically of at least 500.

In one embodiment, the B block provides 10-50% of the weight of the copolymer, while in other embodiments the B block provides 20-40% of the weight of the copolymer, or 25-35% of the weight of the copolymer.

In a preferred embodiment, at least 50% of the ABA or water-insoluble copolymer is biodegradable. In various embodiments, at least 75% of the copolymer is biodegradable, or at least 90% of the copolymer is biodegradable, or essentially all of the copolymer is biodegradable. Preferably, at least 50% of the A block is biodegradable. In various embodiments, at least 75% of the A block is biodegradable; or at least 90% of the A block is biodegradable; or essentially all of the A block is biodegradable.

As used herein, “residues having the structure resulting from the polymerization of” specified monomers refers to the result of the polymerization of those specified chemicals. The same structure may be produced by the polymerization of other monomers and still fall within the scope of the present invention. For instance, a residue of hydroxyacetic acid (HO—CH₂—C(═O)OH) refers to the atoms remaining after hydroxyacetic acid has undergone a homopolymerization reaction so as to form a polyester. In the case of hydroxyacetic acid, such a residue will have the formula —O—CH₂—C(═O)—. In the case of the alkylene oxide, the residue will be an alkylene group joined to an oxygen atom, i.e., —O-alkylene-.

The residue may be formed from the reaction of the specified monomer, or any other monomer which, upon polymerization, affords the same structure. For instance, any of hydroxyacetic acid, the cyclic diester thereof which is commonly referred to as glycolide, a polyester of the formula (—O—CH₂—C(═O)—)ₙ wherein “n” designates the number of repeating units, or a reactive version of hydroxyacetic acid, e.g., hydroxyacetyl chloride, may be used to form the same residue in the A block of the ABA copolymer of the invention.

The triblock copolymers of the invention can be made as a liquid or paste by controlling the molecular weight and adjusting the chemical compositions. These copolymers can be spread on tissue easily due to low viscosity and balanced hydrophilicity. Their degradation rate and drug delivery release rate can also be tailored by proper selection of molecular weight and chemical composition.

Preferably, the liquid or paste ABA triblock copolymers of the invention have a polyalkylene oxide block in the middle (the B block) and two polyester blocks at the ends (the A blocks). Examples of polyalkylene oxide include polyethylene glycol and Pluronics® CDC triblock copolymers from BASF (Parsipanny, N.J.). In the structure CDC, C and D are selected from homopolymers of ethylene oxide and propylene oxide. In certain embodiments of the invention, C is a homopolymer of ethylene oxide and D is a homopolymer of propylene oxide, while in another embodiment C is a homopolymer of propylene oxide and D is a homopolymer of ethylene oxide.

Examples of the polyester include PLA, PGA, PCL and copolymers formed from the corresponding monomers that are used to form PLA, PGA and PCL. The molecular weights of the polyalkylene oxide and the polyester are preferably sufficiently low so as to render the triblock copolymers as a liquid or a paste.

For example, in preferred embodiments of the present invention, block B has a number average molecular weight of less than or equal to 8,000, or less than or equal to 5,000, or less than or equal to 4,000, or less than or equal to 3,000, or less than or equal to 2,000, or less than or equal to 1,000. For each of these specified maximum number average molecular weights, the lower limit of the molecular weight is preferably at least 100, or at least about 500.

In typical copolymers of the invention, the B block provides 10-50% of the weight of the copolymer. In preferred embodiments the B block provides 20-40% of the weight of the copolymer, or provides 25-35% of the weight of the copolymer.

The copolymer is preferably biodegradable in whole or part. For example, in various embodiments the invention provides that at least 50% of the copolymer is biodegradable, or at least 75% of the copolymer is biodegradable, or at least 90% of the copolymer is biodegradable, or essentially all of the copolymer is biodegradable. In addition or alternatively, it is preferred that at least 50% of the A block is biodegradable, or at least 75% of the A block is biodegradable, or at least 90% of the A block is biodegradable, or all or essentially all of the A block is biodegradable.

A preferred polyalkylene oxide is polyethylene glycol with molecular weight equal or less than 4600. A preferred polyester is 50:50 poly(DL-lactide-co-ε-caprolactone) (PLC). A preferred weight ratio of PEG:PLC is 30:70.

The terms “liquid” and “paste” are used in their conventional sense. Thus, a liquid is material which flows, and has a viscosity ranging from that of liquid water to about the viscosity of honey. A paste is a material which will easily deform to a adopt a new shape upon exposure to a shear force, however displays negligible or insignificant change in shape in the absence of shear force, on a timescale of minutes. In contrast, a solid will retain its geometry and does not readily deform under shear force. A “non-solid” has the consistency of a material excepting a solid or gaseous material.

The triblock copolymer may be synthesized through a ring opening bulk melt polymerization procedure. Briefly, monomer PEG (or Pluronics®), DL-lactide, L-lactide, glycolide, and/or ε-caprolactone are added to a reaction vessel. The temperature is raised to 120-180° C. to start the polymerization reaction. A small amount (0.5%) of catalyst (e.g., stannous octoate) is added to accelerate the polymerization. Mixing is performed to ensure homogenicity of the reactants. The polymerization normally takes 2-10 hours.

Alternatively, the water insoluble component of the drug delivery system can be a simple biocompatible polymer. Examples of suitable polymers include polymers of polylactic acid, polyglycolic acid, polycaprolactone, polyanhydride, polybutyric acid, polyacrylic acid, and polymethacrylate. A water insoluble component can also be a copolymer of such polymers. Alternatively, suitable water insoluble polymers can be obtained by forming copolymers comprising (a) hydrophilic polymers, such as polyethylene oxides, polyethylene glycols, pluronics, and polysaccharides, and (b) polymers of polylactic acid, polyglycolic acid, polycaprolactone, polyanhydride, polybutyric acid, polyacrylic acid, and polymethacrylate.

Although the insoluble polymer component of the present invention is a solid at about 37° C., a blend of the insoluble polymer and a soluble polymer is a liquid at about 37° C. Suitable soluble polymers include low molecular weight polyalkylene oxides, such as low molecular weight polyethylene glycol (PEG) and low molecular weight methoxypolyethylene glycol (MePEG). Such soluble polymers are liquid at room temperature. Examples herein illustrates the use of methoxypolyethylene glycol having a number average molecular weight in the range of 100-500, and preferably about 350 in a polymeric drug delivery system. Additional useful soluble polymers include low molecular weight samples of polylactic acid and suitable Pluronic™ polymers.

The insoluble polymer and the soluble polymer are mixed to produce a polymeric blend. A performed method for mixing the water insoluble and water soluble polymers is to warm the polymers to a temperature above the peak melting point of both polymers so that the mixture is liquid. The blend can then stirred using, for example, a spatula. Suitable polymer blends contain insoluble and soluble components in mixtures of about 30: about 70 by weight to about 90: about 10 by weight. Insoluble and soluble components may be present in a polymer blend at a ratio (by weight) of 30:70, 40:60, 50:50, 60:40, 70:30, 80:20, and 90:10.

A polymeric drug delivery system is made by mixing a single polymer, or a polymer blend with a drug to dissolve or to suspend the drug within the blend. The resultant drug delivery system has the form of a liquid or paste at room temperature. The terms “liquid” and “paste” are used in their conventional sense. Thus, a liquid is material which flows, and has a viscosity ranging from that of liquid water to about the viscosity of honey. A paste is a soft, viscous mass of solids dispersed in a liquid, which will easily deform to adopt a new shape upon exposure to a shear force, and displays negligible or insignificant change in shape in the absence of shear force, on a time scale of minutes.

Originally, it was envisaged that, as the water soluble component of a two component polymer blend dissolved out, the water insoluble component would become increasingly waxy and eventually solidify. However, surprisingly, this was found not to occur. Rather, as the water insoluble component dissolved out, the residual insoluble component did not have sufficient structural integrity to remain intact, and the remaining polymer disintegrated into a large number of minute fragments, incompatible with the concept of a slow release drug delivery device. Surprisingly, the present inventors found that an in vivo solidification process requires the use of a hydrophobic drug in the delivery system. Accordingly, after the polymeric drug delivery system of the present invention is placed within a subject, the soluble component of the polymer blend dissolves, resulting in the solidification of the drug and the insoluble polymer to a waxy gel which releases the hydrophobic drug over time.

The amount of drug in a polymeric drug delivery system varies according to the particular drug, the desired therapeutic or prophylactic effect, and the desired duration for which the system is to deliver the drug. In general, the upper limit on the amount of drug included in a polymeric drug delivery system is determined by the need to obtain a suitable viscosity for injection, whereas the lower limit of drug is determined by the activity of the drug and the required duration of treatment. Typically, a polymeric drug delivery system can contain a drug from about 2% to about 30% of the total weight of the system. Preferably, a polymeric drug delivery system contains a hydrophobic drug from about 2.5% to about 20% of the total weight of the system, or from about 2.5% to about 15% of the total weight of the system. For example, a hydrophobic drug can be included in a polymeric drug delivery system at a dose that is 2.5%, 5%, 10%, 15%, 20%, 25%, or 30% of the total weight of the system. Any hydrophobic therapeutic agent can be loaded into the polymeric formulation, as described below.

The polymeric drug delivery system does not require any pre-injection mixing. If necessary, the system can be sterilized by gamma radiation, and stored for long periods without compromise in properties.

It is possible to control the in vivo degradation rate of the insoluble component and drug by varying the molecular weight of the insoluble polymer. Moreover, the in vivo solidification process and rate of drug release can be controlled by altering the amount of drug in the polymeric drug delivery system. As an illustration, the examples show that a drug delivery system comprising 2.5% of a drug forms a less solid mass and releases drug at a quicker rate than a comparable system having 15% of the same drug. This feature is a great advantage over existing injectable pastes, which do not have control of these properties.

In general terms, the degree of solidification is increased with increasing amounts of drug in the composition. A more solid mass would decrease the release rate of drug from the blend relative to a less solid mass because the more solid mass would be less deformable, so that any drug encapsulated in interior zones of the solid mass would have farther to travel to the exterior zones (i.e., the area of drug release). Furthermore, a less solid mass would tend to fragment more easily, increasing the surface area of the solid and allowing faster drug release. This variation in the degree of disintegration also affects the degradation rate of the solid mass, because the increased surface area of a disintegrated mass allows more exposure to water and more exposure to immune responses, such as phagocytosis, elicited by the host.

With regard to the amount of water soluble polymer in the blend, when more water soluble polymer is added to the blend, the release rate of the drug from the blend increases and the degree of disintegration of the blend increases. The reason for these effects is that the dissolution of the water soluble component from the high ratio blends causes the residual water insoluble component to solidify with insufficient structural integrity due to the low amount of insoluble polymer remaining. Such a mass disintegrates easily, increasing the surface area, allowing faster drug release and faster degradation.

In summary, faster drug release rates and degradation rates may be achieved by using lower drug loadings and/or higher ratios of water soluble to water insoluble polymers in the blend. Slower drug release rates may be achieved using high drug loadings and lower ratios of water soluble to insoluble polymers in the blend. These factors allow for control of dose in a rather unique manner.

The effect of drug loading on release rate was unexpected because the opposite effect was predicted based on the theory of drug release rates from polymeric implants that is accepted by those skilled in the art. See, e.g., Desai et al., J. Pharm. Sci. 55:1224 (1966). That is, higher drug loadings were predicted to lead to faster drug release rates because released drug might allow water to penetrate deeper regions of the polymeric mass to dissolve (and release) more drug. This unexpected feature of the presently described composition offers great advantages over existing paste formulations.

For example, for the treatment of diseases where a short burst of drug release (e.g., one week) at a relatively high concentration is required, one might select a low drug loading in the polymeric blend. If a physician needed very low concentrations of drug over extended periods of time, then the physician might inject a small amount of a high drug loaded paste which released drug more slowly.

As an illustration, a physician may wish to treat two diseases with paclitaxel, prostate cancer and restenosis due to angioplasty. To treat cancer, it would be preferable to achieve a very slow release of drug (e.g., 20 μg/day) for three months, but for restenosis, the physician might require 200 μg/day for just 2 weeks. Using a known paste formulation containing polycaprolactone, as described, for example, by Winternitz et al., Pharm. Res. 13:195 (1996), it might be possible to achieve a 20 μg/day release for three months with an injection of about 200 mg of 10% paclitaxel loaded polycaprolactone paste. The total amount of paclitaxel injected as a depot would be just 20 mg and therefore far below the toxic limit of 200 mg as a single dose. However, an attempt to achieve the 200 μg/day dose required for restenosis using this paste would present many problems. First, it would be necessary to inject either 2000 mg of 10% paclitaxel loaded paste or about 1000 mg of 20% paclitaxel loaded paste. This large amount of paste would be extremely difficult to locate at the site of restenosis. Moreover, it would require the injection of a total amount of paclitaxel (200 mg) that exceeded the toxic limit for a single dose in humans. In addition, this approach would result in the release of 200 μg/day for many months (not just two weeks) which could induce cytotoxicity in normal cells at the restenosis site. Clearly, the polycaprolactone paste does not allow for controlled drug release to fit different treatment needs since a higher daily dose can only be achieved by using more paste or a higher drug loading. The advantage of the paste described herein is that a high dose over a short period may be achieved using a lower drug loading.

In addition to the effect of drug loading on both the rate and duration of drug release, the duration and rate of drug release may be further controlled by modulating the ratio of water soluble polymer to water insoluble polymer. Therefore, by careful selection of the ratio of drug:water soluble polymer:water insoluble polymer, the paste may be tuned to fit the required treatment needs.

The polymeric composition may be injected into sensitive areas such as the synovial joint or adjacent to nerves where a more wax-like gel would be beneficial. Reducing the initial drug concentration allows for this wax-like gel property. Alternatively, if a very slow release system was required and a more solid and permanent implant was needed (e.g., for a paste to cover a tumor resection site), a composition can be designed with a high initial drug loading that solidifies quickly and releases drug slowly.

The present invention does not require the use of organic solvents for dissolving the drugs during manufacturing nor for solidification of the implant. As used herein, the term “organic solvent” refers to non-polymeric substances, such as aromatic hydrocarbons, esters, ethers, ketones, amines, alcohols, nitrated hydrocarbons, and chlorinated hydrocarbons. For example, solvents that are typically used in polymer drug delivery systems include acetone, ethanol, tetrahydrofuran and pyrrolidones. Since these compounds are not biocompatible, they are not suitable for in vivo injection into delicate areas such as the eye, blood vessels, or the synovial joint.

Another advantage of the present invention is that the drug-loaded implant is deformable because the major component is a wax at 37° C. The degree of solidification increases very slowly with time as the water-soluble polymer dissolves out and the drug precipitates in the triblock. This feature allows the implant to mold more precisely to the required site without the sharp or brittle edges that might form with other polymer implants. Accordingly, this characteristic makes the presently described compositions particularly advantageous for injection into sensitive body tissues.

For example, after a 40:60 triblock copolmer:methoxypolyethylene glycol (TB:MePEG) 10% paclitaxel composition was injected into a subcutaneous tumor in nude mice, the paste was observed to maintain a gel-like transparent nature for two to three hours. The paste then became opaque (due to paclitaxel precipitation) and took a more solid form. However, the paste-implant was still deformable after 24 hours.

As mentioned above, the solidification process of the polymeric drug delivery system having both a water soluble and water insoluble polymer component requires the presence of a hydrophobic drug. Compositions that only comprise the two polymers in any ratio (but no drug) may gel when placed in water at 37° C. but the gel disperses quickly and the solid implant never forms. The solidification process occurs only when hydrophobic drugs are dissolved into the polymeric blend. Hydrophilic drugs do not have the same characteristics that allow the polymeric drug delivery system to solidify. However, the hydrophilic drugs may be used in certain triblock polymers of the invention, and specifically those that are a triblock copolymer of the formula ABA, wherein A is a block of residues that includes the residues which remain after polymerization of one or more monomers selected from hydroxyacetic acid, 2-hydroxypropionic acid and 6-hydroxyhexanoic acid, B is a block of residues that includes the residues which remain after the polymerization of one or more monomers selected from alkylene oxide and alkylene glycol, where the copolymer is either a paste or liquid at a temperature within the range of 25-40° C., or has a non-solid consistency at 25° C.

The present invention provides non-solid, preferably liquid or paste polymers which may be injected into a subject at or near physiological temperatures. Alternatively, these polymers may be spread onto tissue. These polymers may also degrade and release a bioactive ingredient, e.g., paclitaxel, rapidly. The polymers of the invention may be made biodegradable.

3. Therapeutic Uses of Polymeric Drug Delivery Systems

The polymeric drug delivery systems described herein can be used to deliver either a hydrophobic or (dependent on the drug delivery system) a hydrophilic drug in controlled manner either to a localized site or to the systemic circulation. Examples of diseases that may be treated this way and drugs that may be used for such diseases are described below.

Although the discussion below focuses on localized delivery of drugs to treat such diseases as cancer, arthritis and restenosis, the polymeric drug delivery system can also allow for the implantation of solid implants that provide a controlled release of drugs for systemic absorption over a period of weeks or months, by the relatively noninvasive method of injecting the patient, rather than by the more traditional invasive surgery that to date is required for implantation of solid implants. This approach should decrease cost, patient discomfort and non-compliance that is associated with traditional invasive implantation by surgery or multiple injections of drugs or multiple oral administration of drugs.

For example, many insulin dependent diabetic patients require multiple subcutaneous injections of various types of insulin daily for systemic absorption. The polymeric drug delivery system suitable for hydrophobic drugs of the present invention can provide for the controlled release of insulin over a week or a month from a single subcutaneous depo. Human insulin is practically insoluble in water from pH 4.5 to 7.0. See, for example, Windholz, (Ed.), The Merck Index, 10th Edition, (Merck & Co., Inc. 1983) [“The Merck Index”].

Many hydrophobic drugs are converted to water soluble salts of the drug by pharmaceutical companies to enable easy uptake into the blood stream from the gut or for aqueous solutions of such drugs to be injected into the body. However, the water soluble forms of these drugs are usually cleared from the body rapidly. The advantage of a polymeric paste injection formulation is that the original pure form of the drug (non-salt, hydrophobic form) may be used. For example, cromoglycate is used for the treatment of local allergic reactions and symptoms including periopthalmic and inside the eyelid or intranasal application. Cromoglycate is an anti-allergy agent and is currently available as a sodium salt. Levobunolol is used for the treatment of glaucoma by peri-ophthalmic and inside the eyelid application. Levobunolol is a beta blocker and is currently available as the hydrochloride salt. Terbinafine is used for the treatment of a skin or nail fungal infection by injection subcutaneously below the fungal infection. Terbinafine is an antifungal and is currently available as the hydrochloride salt.

Examples of diseases that may be treated with a polymeric drug delivery system include cancer, bacterial infections, psoriasis, arthritis and other inflammatory conditions, fungal infections, vascular disease, ocular disease and diabetes. The polymeric drug delivery system can be administered to a patient by intraperitoneal, intraarticular, intraocular, intratumoral, perivascular, subcutaneous, intracranial or intramuscular injection. A polymeric drug delivery system may also be administered by application to mucus membranes, including periophthalmic and inside the eyelid, intraoral, intranasal, intrabladder intravaginal, intraurethral, intrarectal and to the adventitia of an internal organ.

In addition to neoplastic or proliferative diseases, other diseases such as vascular disease can result in the narrowing, weakening and/or obstruction of body passageways. According to 1993 estimates (source—U.S. Heart and Stroke Foundation homepage), over 60 million Americans have one or more forms of cardiovascular disease. These diseases claimed 954,138 lives in the same year (41% of all deaths in the United States).

Balloon angioplasty (with or without stenting) is one of the most widely used treatments for the vascular disease; other options such as laser angioplasty are also available. While this is the treatment of choice in cases of severe narrowing of the vasculature, about one-third of patients undergoing balloon angioplasty (source—Heart and Stoke Foundation homepage, http://www.hsfope.org) have renewed narrowing of the treated arteries (restenosis) within 6 months of the initial procedure; often serious enough to necessitate further interventions.

Such vascular diseases (including for example, restenosis) are due at least in part to intimal thickening secondary to vascular smooth muscle cell (VSMC) migration, VSMC proliferation, and extra-cellular matrix deposition. Briefly, vascular endothelium acts as a nonthrombogenic surface over which blood can flow smoothly and as a barrier which separates the blood components from the tissues comprising the vessel wall. Endothelial cells also release heparin sulphate, prostacyclin, EDRF and other factors that inhibit platelet and white cell adhesion, VSMC contraction, VSMC migration and VSMC proliferation. Any loss or damage to the endothelium, such as occurs during balloon angioplasty, atherectomy, or stent insertion, can result in platelet adhesion, platelet aggregation and thrombus formation. Activated platelets can release substances that produce vasoconstriction (serotonin and thromboxane) and/or promote VSMC migration and proliferation (PDGF, epidermal growth factor, TGF- and heparinase). Tissue factors released by the arteries stimulates clot formation resulting in a fibrin matrix into which smooth muscle cells can migrate and proliferate.

This cascade of events leads to the transformation of vascular smooth muscle cells from a contractile to a secretory phenotype. Angioplasty induced cell lysis and matrix destruction results in local release of basic fibroblast growth factor (bFGF) which in turn stimulates VSMC proliferation directly and indirectly through the induction of PDGF production. In addition to PDGF and bFGF, VSMC proliferation is also stimulated by platelet released EGF and insulin growth factor-1.

Vascular smooth muscle cells are also induced to migrate into the media and intima of the vessel. This is enabled by release and activation of matrix metalloproteases which degrade a pathway for the VSMC through the extra-cellular matrix and basement membrane. After migration and proliferation the vascular smooth muscle cells then deposit extra-cellular matrix consisting of gylcosaminoglycans, elastin and collagen which comprises the largest part of intimal thickening. A significant portion of the restenosis process may be due to remodeling leading to changes in the overall size of the artery; at least some of which is secondary to proliferation within the adventitia (in addition to the media).

In summary, virtually any forceful manipulation within the lumen of a blood vessel will damage or denude its endothelial lining. Thus, treatment options for vascular diseases themselves and for restenosis following therapeutic interventions continue to be major problems with respect to long-term outcomes for such conditions.

In addition to neoplastic obstructions and vascular disease, there are also a number of acute and chronic inflammatory diseases which result in obstructions of body passages. These include, for example, vasculitis, gastrointestinal tract diseases and respiratory tract diseases.

Each or these diseases can be treated, to varying degrees of success, with medications such as anti-inflammatories or immunosuppressants. Current regimens however are often ineffective at slowing the progression of disease, and can result in systemic toxicity and undesirable side effects. Surgical procedures can also be utilized instead of or in addition to medication regimens. Such surgical procedures however have a high rate of local recurrence to due to scar formation, and can under certain conditions (e.g., through the use of balloon catheters), result in benign reactive overgrowth.

4. Treatment or Prevention of Disease

As noted above, the present invention provides methods for treating or preventing a wide variety of diseases associated with the obstruction of body passageways, including for example, vascular diseases, neoplastic obstructions, inflammatory diseases, and infectious diseases.

For example, within one aspect of the present invention a wide variety of therapeutic compositions as described herein may be utilized to treat vascular diseases that cause obstruction of the vascular system. Representative examples of such diseases include artherosclerosis of all vessels (around any artery, vein or graft) including, but not restricted to: the coronary arteries, aorta, iliac arteries, carotid arteries, common femoral arteries, superficial femoral arteries, popliteal arteries, and at the site of graft anastomosis; vasospasms (e.g., coronary vasospasms and Raynaud's Disease); restenosis (obstruction of a vessel at the site of a previous intervention such as balloon angioplasty, bypass surgery, stent insertion and graft insertion).

Briefly, in vascular diseases such as atherosclerosis, white cells, specifically monocytes and T lymphocytes adhere to endothelial cells, especially at locations of arterial branching. After adhering to the endothelium, leukocytes migrate across the endothelial cell lining in response to chemostatic stimuli, and accumulate in the intima of the arterial wall, along with smooth muscle cells. This initial lesion of athersosclerosis development is known as the “fatty streak”. Monocytes within the fatty streak differentiate into macrophages; and the macrophages and smooth muscle cells progressively take up lipids and lipoprotein to become foam cells.

As macrophages accumulate, the overlying endothelium becomes mechanically disrupted and chemically altered by oxidized lipid, oxygen-derived free radicals and proteases which are released by macrophages. Foam cells erode through the endothelial surface causing micro-ulcerations of the vascular wall. Exposure of potentially thrombogenic subendothelial tissues (such as collagen and other proteins) to components of the bloodstream results in adherence of platelets to regions of disrupted endothelium. Platelet adherence and other events triggers the elaboration and release of growth into this mileau, including platelet-derived growth factor (PDGF), platelet activating factor (PAF), and interleukins 1 and 6 (IL-1, IL-6). These paracrine factors are thought to stimulate vascular smooth muscle cell (VSMC) migration and proliferation.

In the normal (non-diseased) blood vessel wall, vascular smooth muscle cells have a contractile phenotype and low index of mitotic activity. However, under the influence of cytokines and growth factors released by platelets, macrophages and endothelial cells, VSMC undergo phenotypic alteration from mature contractile cells to immature secretory cells. The transformed VSMC proliferate in the media of the blood vessel wall, migrate into the intima, continue to proliferate in the intima and generate large quantities of extracellular matrix. This transforms the evolving vascular lesion into a fibrous plaque. The extracellular matrix elaborated by secretory VSMC includes collagen, elastin, glycoprotein and glycosaminoglycans, with collagen comprising the major extracellular matrix component of the atherosclerotic plaque. Elastin and glycosaminoglycans bind lipoproteins and also contribute to lesion growth. The fibrous plaque consists of a fibrous cap of dense connective tissue of varying thickness containing smooth muscle cells and macrophages overlying macrophages, T cells and extracellular material.

In addition to PDGF, IL-1 and IL-6, other mutogenic factors are produced by cells which infiltrate the vessel wall including: transforming growth factor beta (TGF-??, fibroblast growth factor (FGF), thrombospondin, serotonin, thromboxane A₂, norepenephrine, and angiotension II. This results in the recruitment of more cells, elaboration of further extracellular matrix and the accumulation of additional lipid. This progressively enlarges the atherosclerotic lesion until it significantly encroaches upon the vascular lumen. Initially, obstructed blood flow through the vascular tube causes ischemia of the tissues distal to the atherosclerotic plaque only when increased flow is required—later as the lesion further blocks the artery, ischemia occurs at rest.

Macrophages in the enlarging atherosclerotic plaque release oxidized lipid, free radicals, elastases, and collageneses that cause cell injury and necrosis of neighbouring tissues. The lesion develops a necrotic core and is transformed into a complex plaque. Complex plaques are unstable lesions that can: break off causing embolization; hemorrhage locally (secondary to rupture of the vasa vasora supplying the plaque which results in obstruction of the lumen due to rapid expansion of the lesion); or ulcerate and fissure to expose the thrombogenic necrotic core to the blood stream producing local thrombosis or distal embolization. Even should none of the above sequela occur, the adherent thrombus may become organized and incorporated into the plaque thereby accelerating its growth. Furthermore, as the local concentrations of fibrinogen and thrombin increase, proliferation of vascular smooth muscle cells within the media and intima is stimulated; a process which also ultimately leads to additional narrowing of the vessel.

The intima and media of normal arteries are oxygenated and supplied with nutrition from the lumen of the artery or from the vasa vasorum in the adventitia. With the development of atherosclerotic plaque, microvessels arising from the adventitial vasa vasorum extend into the thickened intima and media. This vascular network becomes more extensive as the plaque worsens and diminishes with plaque regression.

Hemorrhage from these microvessels may precipitate sudden expansion and rupture of plaque in association with arterial dissection, ulceration, or thrombosis. It has also been postulated that the leakage of plasma proteins from these microvessels may attract inflammatory infiltrates in the leaked plasma proteins and infiltrated in inflammatory cells may contribute to the rapid growth of atherosclerotic plaque and to associated complications through edema and inflammation.

5. Formulation and Administration

As noted above, therapeutic compositions of the present invention may be formulated in a variety of forms (e.g., microspheres, pastes, films or sprays). Further, the compositions of the present invention may be formulated to contain more than one therapeutic agents, to contain a variety of additional compounds, to have certain physical properties (e.g., elasticity, a particular melting point, or a specified release rate). Within certain embodiments of the invention, compositions may be combined in order to achieve a desired effect (e.g., several preparations of microspheres may be combined in order to achieve both a quick and a slow or prolonged release of one or more anti-angiogenic factor).

Therapeutic agents and compositions of the present invention may be administered either alone, or in combination with pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.

As noted above, therapeutic agents, therapeutic compositions, or pharmaceutical compositions provided herein may be prepared for administration by a variety of different routes, including for example, directly to a body passageway under direct vision (e.g., at the time of surgery or via endoscopic procedures) or via percutaneous drug delivery to the exterior (adventitial) surface of the body passageway (perivascular delivery). Other representative routes of administration include gastroscopy, ECRP and colonoscopy, which do not require full operating procedures and hospitalization, but may require the presence of medical personnel.

Briefly, perivascular drug delivery involves percutaneous administration of localized (often sustained release) therapeutic formulations using a needle or catheter directed via ultrasound, CT, fluoroscopic, MRI or endoscopic guidance to the disease site. Alternatively the procedure can be performed intra-operatively under direct vision or with additional imaging guidance. Such a procedure can also be performed in conjunction with endovascular procedures such as angioplasty, atherectomy, or stenting or in association with an operative arterial procedure such as endarterectomy, vessel or graft repair or graft insertion.

For example, within one embodiment polymeric paclitaxel formulations can be injected into the vascular wall or applied to the adventitial surface allowing drug concentrations to remain highest in regions where biological activity is most needed. This has the potential to reduce local “washout” of the drug that can be accentuated by continuous blood flow over the surface of an endovascular drug delivery device (such as a drug-coated stent). Administration of effective therapeutic agents to the external surface of the vascular tube can reduce obstruction of the tube and reduce the risk of complications associated with intravascular manipulations {such as restenosis (see next), embolization, thrombosis, plaque rupture, and systemic drug toxicity}.

For example, in a patient with narrowing of the superficial femoral artery, balloon angioplasty would be performed in the usual manner (i.e., passing a balloon angioplasty catheter down the artery over a guide wire and inflating the balloon across the lesion). Prior to, at the time of, or after angioplasty, a needle would be inserted through the skin under ultrasound, fluoroscopic, or CT guidance and a therapeutic agent (e.g., paclitaxel impregnated into a slow release polymer) would be infiltrated through the needle or catheter in a circumferential manner directly around the area of narrowing in the artery. This could be performed around any artery, vein or graft, but ideal candidates for this intervention include diseases of the carotid, coronary, iliac, common femoral, superficial femoral and popliteal arteries and at the site of graft anastomosis. Logical venous sites include infiltration around veins in which indwelling catheters are inserted.

The therapeutic agents, therapeutic compositions and pharmaceutical compositions provided herein may be placed within containers, along with packaging material which provides instructions regarding the use of such materials. Generally, such instructions include a tangible expression describing the reagent concentration, as well as within certain embodiments, relative amounts of excipient ingredients or diluents (e.g., water, saline or PBS) which may be necessary to reconstitute the anti-angiogenic factor, anti-angiogenic composition, or pharmaceutical composition.

Pharmaceutical formulation can be prepared by loading therapeutic agents into the triblock copolymers and/or the polymeric blends. The loading can be done by mixing drug directly into the copolymer or by co-dissolving both drug and the copolymer in a common organic solvent (e.g., acetonitrile, dichloromethane) followed by solvent removal using evaporation and/or vacuumization. The second approach is preferred for loading paclitaxel into the ABA triblock copolymers since it ensures homogenicity and a composition that affords fast release of paclitaxel.

Any therapeutic agent can be loaded into the ABA triblock copolymers (in contrast to the polymeric blends, which require a hydrophobic drug). Examples of the agents include, without limitation, peptides, proteins, antigens, vaccines, anti-infectives, antibiotics, antimicrobials, antiallergenics, steroids, decongestants, miotics, anticholinergios, sympathomimetics, sedatives, hypnotics, psychic energizers, tranquilizers, analgesics, antimalarials, and antihistamines.

Thus, the present invention provides liquid or paste triblock copolymers of PE-PEG-PE for medical applications. The liquid and paste copolymers may be obtained by employing low molecular weight PEG and PE, and by using random PE copolymers consisting of ε-caprolactone. The invention also provides drug delivery systems using the triblock copolymers, which can be easily administered and give rapid drug release and polymer degradation. The delivery systems of the invention have the improved properties of: injectability, spreadability on tissues, rapid degradation and fast release of drugs such as paclitaxel.

Examples of hydrophobic drugs that could be used in this polymeric drug delivery system, or with the ABA triblock copolymers, include the following.

Amphotericin can be used for the treatment or prevention of infection of an open wound by topical administration or for the treatment or prevention of an infection in an exposed wound after surgery by local application. Amphotericin is an antifungal and is insoluble in water at pH 6 to 7. See, e.g., The Merck Index.

Anthralin can be used for the treatment of “wet” psoriasis by topical application. Anthralin is an agent for psoriasis therapy and is practically insoluble in water. See, e.g., The Merck Index.

Beclomethasone can be used for the reduction of local inflammation by peri-ophthalmic and inside the eyelid or intranasal (e.g., for the treatment of rhinitis) application. Beclomethasone is a corticosteroid and is very slightly soluble in water. See, for example, Gennaro, (ed.), Remington's Pharmaceutical Sciences, 17th Edition, (Mack Publishing Company 1985).

Betamethasone is used for the reduction of local inflammation by oral (e.g., canker sore), intravaginal, and intrarectal application. Betamethasone is a corticosteroid and has a solubility of 190 μg/mL water. See, for example, Gennaro, (ed.), Remington's Pharmaceutical Sciences, 17th Edition, (Mack Publishing Company 1985).

Camptothecin is used for the treatment of diseases involving cellular proliferation such as cancer, arthritis, psoriasis, restenosis, surgical adhesions. Camptothecin has a water solubility of 1-2 μg/mL.

Curcumin is a potent antioxidant and is under investigation as an anti-arthritic drug. Curcumin is practically insoluble in water.

Dexamethasone is used for the reduction of local inflammation by oral application (e.g., post wisdom tooth removal). Dexamethasone is a corticosteroid and has a solubility of 10 μg/mL in water. See, e.g., The Merck Index.

Indomethacin is used for the treatment of symptoms of gout by intraarticular or intramuscular injection, or for the reduction of local inflammation by peri-ophthalmic and inside the eyelid, oral, intranasal, intravaginal and intrarectal application. Indomethacin is a non-steroidal anti-inflammatory (NSAID) and is practically insoluble in water. See, e.g., The Merck Index.

Genistein is a tyrosine kinase inhibitor and is under investigation for the treatment of diseases involving cellular proliferation. Genistein is practically insoluble in water.

Lidocaine provides local anesthesia by intramuscular injection, or administration by application to mucus membranes, including periophthalmic and inside the eyelid, oral, intranasal, intravaginal and intrarectal. Lidocaine is a local anesthetic and is practically insoluble in water. See, for example, Gennaro, (ed.), Remington's Pharmaceutical Sciences, 17th Edition, (Mack Publishing Company 1985).

Proteins that are practically insoluble in water, such as insulin, can be used in the presently described polymeric drug delivery system.

Paclitaxel is used for the treatment of angiogenic related diseases such as arthritis, cancer, restenosis, psoriasis, or surgical adhesions. Paclitaxel has a water solubility of 1-2 μg/mL.

Tetracycline is used for the treatment of eye infections by peri-ophthalmic and inside the eyelid application. Tetracycline is an antibacterial and has a solubility of 400 μg/mL water. See, e.g., Gennaro, (ed.), Remington's Pharmaceutical Sciences, 17th Edition, (Mack Publishing Company 1985).

Tretinoin is a retinoic acid that is being investigated as an anticancer agent. Tretinoin is practically insoluble in water.

The drug delivery systems of liquid or paste triblock copolymers can be used to treat any diseases where applicable. For example, when paclitaxel is loaded into the copolymer, the formulation can be: injected directly into a solid tumor to treat cancer; applied to a tumor resection cavity to kill the residual cancer cells; spread on tissues to prevent post-surgical adhesion; applied perivascularly to treat restenosis; or injected intra-articularly to treat arthritis.

As an illustration of the application of the present invention, the drug delivery systems are well-suited for treatment of prostate cancer. Prostate cancer is the most common cancer and the second highest cause of cancer death in men (Carter et al., Prostate, 16:39-48, 1990). Due to increased public awareness and diagnosis of the disease, the reported incidence of prostate cancer continues to rise each year (Scher, Seminars in Oncology, 21:511-513, 1994). Furthermore, with the prospect of the projected aging of the American population, it is likely that even more cases will appear in the future (Colombel et al., Am. J. Pathol., 143:390-400, 1993). Unfortunately, prostate cancer morbidity is reported to be increasing continuously, or is at best leveling off despite earlier detection of the disease (Scher, Seminars in Oncology, 21:511-513, 1994).

For patients presenting with localized prostate tumors, a number of aggressive therapeutic options are available. Some patients require radical prostasectomy, some require aggressive radiotherapy and/or aggressive chemotherapy. A significant portion of patients treated with radiotherapy fail to respond fully with local recurrence of the prostate tumor. Therefore, patients with recurring localized tumors, or patients with localized tumors who are not candidates for aggressive therapy, would benefit from additional local treatment modalities.

Patients with prostate cancer may present in different stages of the disease so that patients in early stages may have localized lesions only, whereas in advanced disease states, patients may also have metastatic disease that, in turn, may be either androgen dependent or androgen independent. Although most patients have androgen dependent metastatic disease, the size of this patient group is dwarfed by the number of men with localized but non-symptomatic disease. At least 30% of men over 50 years of age have histological evidence of localized prostate cancer yet most of these cancers remain undetected or become a problem during the lifetime of these men (Guileyardo et al., J. Natl. Cancer Inst., 65:311-317, 1980; Wasson et al., Arch. Fam. Med., 2:487-493, 1993; Franks, Cancer, 32:1092-1095, 1973). Although routine screening of asymptomatic men will undoubtedly increase the detection of localized tumors it is not known whether early detection will increase survival rates, especially as many physicians advise a “no therapy” approach to patients with localized tumors.

While this approach does little to satisfy the patient who expects an aggressive treatment for the malignancy (Scher, Seminars in Oncology, 21:511-513, 1994), there is justification for not adopting an aggressive treatment regimen since conservative management and delayed hormone therapy treatment of localized tumors has been shown to be as effective a treatment as radical surgical removal of tumors (Chodak et al., N. Engl. J. Med., 330:242-248, 1994; Madson et al., Scand. J. Urol. Nephrol. Suppl., 110:95-100, 1988). Clearly, alternative chemotherapeutic methods are needed for patients with localized prostate cancer to prevent metastatic progression of the disease and to offer the patient a non-invasive treatment of the tumor.

A more rational approach to the administration of a drug for the treatment of localized prostate tumors can be provided by a slow release implant device that could deliver chemotherapeutically relevant doses of a drug to the tumor site. Such a formulation might avoid the systemic toxicity problems associated with repeated treatment regimens. The prostate gland is amenable to local injection (Reft Broading therapy) and thus a single injection of a drug-loaded polymeric paste formulation administered intra-tumorally into human prostate tumors may be efficacious.

At present, there are no effective chemotherapeutic agents for the treatment of prostate cancer, although drugs such as estramustine and vinblastine, which also inhibit microtubule function, have shown some efficacy against prostate cancer both in vitro (Speicher et al., Cancer Res., 52:4433-4440, 1992; Darby et al., Anticancer Res., 16:3647-3652, 1996; Spencer et al., Drugs, 48:794-847, 1994) and in vivo (Spencer et al., Drugs, 48:794-847, 1994; Seidman et al., J. Urol., 147:931-934, 1992; Pienta et al., Cancer, 75:1920-1926, 1995).

Paclitaxel has also been reported to inhibit human prostate cancer cell growth in vitro (Speicher et al., Cancer Res., 52:4433-4440, 1992; Halder et al., Cancer Res., 56:1253-1255, 1992; Darby et al., Anticancer Res., 16:3647-3652, 1996). Moreover, paclitaxel has been shown to have a potent inhibitory effect on angiogenesis (Oktaba et al., AACR 36:454, 1995), a process that has been proposed as a target for the chemotherapeutic treatment of prostate cancer.

Although angiogenesis is associated with tumor growth in all types of cancer, this process may have particular relevance to prostate cancer. Post-mortem studies have shown that up to 30% of all removed prostates have latent prostate cancer (Guileyardo et al., J. Natl. Cancer Inst. 65:311-317, 1980) in which clinically non-apparent carcinomas may be at a prevascular (and slow growing) phase due to the lack of sufficient angiogenic phenotypes in the tumor mass (Furusato et al., Br. J. Cancer 70:1244-1246, 1994). Furthermore, increased angiogenic activity is also associated with metastatic disease in prostate cancer, and it has been suggested that specific inhibition of angiogenesis might inhibit the development of metastasis (Vukanovic et al., The Prostate 26:235-246, 1995). Indeed, a treatment based on the use of the antiangiogenic drug Linomide has been shown to have both antitumor and antimetastatic effects against prostate tumors grown in rats via inhibition of angiogenesis (Vukanovic et al., The Prostate 26:235-246, 1995).

Therefore, with early detection of prostate cancer, the inhibition of angiogenesis may provide an effective “holding” therapy for many patients with localized tumors. Paclitaxel may therefore provide a particularly useful agent in the treatment of prostate cancer via the induction of tumor cell apoptosis and through the inhibition of tumor angiogenesis.

Studies have been conducted to assess the use of biocompatible, biodegradable polymeric pastes for the site-directed delivery of antineoplastic agents such as paclitaxel (Winternitz et al., Pharm. Res. 13:368-375, 1996) or bis(maltolato)oxovanadium (Jackson et al., Br. J. Cancer 75:1014-1020, 1997). These surgical pastes were originally designed as an adjunct to tumor resection therapy whereby a residual slow release formulation of the drug would be applied to the resection site to prevent tumor regrowth. Such pastes were composed of polycaprolactone blended with methoxypolyethylene glycol and were applied as a viscous molten paste at 56° C., setting to a solid drug-polymer implant at body temperature. However, the paste was very difficult to inject, due to the viscosity of the polymer, and some large tumors failed to respond fully to the drug implant, probably due to the very slow release characteristics of the formulation (Winternitz et al., Pharm. Res. 13:368-375, 1996). Hence, there was a failure to achieve a chemotherapeutically effective dose. The present invention provides chemotherapeutically effective doses of one or more drugs.

To date, all chemotherapeutic treatments of prostate cancer have palliative goals so that cure has been a rare feature of any trials (Carducci et al., Seminars in Oncology 23(6) Suppl. 14:56-62, 1996). Generally, a strategy of conservative management and delayed hormone therapy is advised for men with localized prostate cancer, especially if the life expectancy of the patient is less than ten years (Chodak et al., N. Engl. J. Med. 330:242-248, 1994). Paclitaxel-loaded polymers can serve in the effective, non-invasive treatment of localized prostate cancer, which offers a cure rather than a holding therapy for patients of all ages with localized prostate cancer.

In addition to prostate cancer, paclitaxel has shown efficacy against advanced breast, ovarian and non-small cell lung cancer (Spencer et al., Drugs, 48:794-847, 1994). Thus, polymeric drug delivery devices containing paclitaxel can also be used to treat these neoplastic conditions.

As mentioned above, additional preferred drugs for inclusion in polymeric delivery systems include camptothecin, amphoterecin, nystatin, tretinoin, genistein, and curcumin. Camptothecin is also an insoluble drug in aqueous solutions. Camptothecin is an anti-proliferative, anti-cancer, anti-viral compound that binds to the DNA-Topol complex resulting in the inhibition of topoisomerase I leading to the inhibition of DNA synthesis. Camptothecin also inhibits the synthesis of ribosomal RNA and thus affects protein synthesis.

The anti-fungal drugs, amphoterecin and nystatin, increase the permeability of cell membranes and induce lysis. These agents are also cytolytic to other cells and cause hemolysis if given systemically at high concentrations. Since these two agents are very hydrophobic, there are serious formulation problems. That is, the compounds must be reconstituted as suspensions in water or saline due to low water solubility. Although these drugs can be used both locally and systemically, local delivery is preferred due to its systemic toxicity.

Tretinoin is a retinoic acid marketed as Retin-A (Registered in the name of McNeil Pharmaceutical Ltd.) for the treatment of acne. This agent increases membrane permeability in certain cell particles; the lysosomes, thereby releasing certain enzymes which may inhibit keratin formation and mucous metaplasia. Retinoic acid is also a cell-differentiating agent and is being explored as an anticancer agent.

Genistein is a tyrosine kinase inhibitor which leads to the inhibition of cell activation. Tyrosine kinases are signaling enzymes that promote cell surface receptors to transmit activation signals into the cell. Therefore, genistein may be used as an anti-inflammatory, anti-proliferative, anti-angiogenic or anticancer agent. Since this agent may detrimentally affect all cells systemically, localized application of slow release forms at disease sites may be beneficial.

Curcumin is a potent antioxidant found in the oriental spice turmeric and has been used in folklore medicine for centuries for many indications. Curcumin has hypolipedemic and hypocholesterolemic properties. It inhibits the generation and release of proinflammatory agents such as superoxide, hydrogen peroxide and nitric oxide and lowers the production of prostaglandins and leukotrienes. These features would support the anti-arthritic action of curcumin in animals and humans.

The polymeric drug delivery systems described herein can be injected through various gauge needles depending on the ratio of insoluble to water soluble polymer. Compositions comprising 40:60 TB:MePEG polymer blends with 15% drug loading, for example, can be injected through 22- or 23-gauge needles at room temperature, allowing access to all body compartments. These injectable properties are not dependent on predissolving the composition in solvents such as N-methyl-pyrrolidone.

The formulation does not require thermal modification for injection or solidification, and consequently, polymeric compositions can be injected at room temperature through narrow gauge needles without blocking. Nevertheless, lower viscosity and improved injectability may be attained by warming the polymeric formulation to 37° C. prior to injection. This will allow the paste to be injected through smaller gauge needles for more delicate tissue areas.

A polymeric drug delivery system (containing a blend of water insoluble and water soluble polymer components with a hydrophobic drug(s)) or a drug in combination with an ABA triblock copolymer (in total, referred to as polymeric compositions, or drug delivery systems), can be administered to a subject by intraperitoneal, intraarticular, intraocular, intratumoral, perivascular, subcutaneous, intracranial, or intramuscular injection. Alternatively, the polymeric compositions can be applied to surgically exposed tissue areas by using an open syringe to extrude the polymeric paste at room temperature. For example, a polymeric composition loaded with paclitaxel can be: (a) injected directly into a solid tumor to treat cancer, (b) applied to a tumor resection site to prevent local recurrence, (c) spread on tissues to prevent post-surgical adhesions, (d) applied perivascularly to treat restenosis, and/or (e) injected intra-articularly to treat arthritis.

The polymeric compositions described herein may also be used to fill the cavities of bones. In such orthopedic or dental applications, the hydrophobic component may be a drug such as a corticosteriod. Alternatively, the hydrophobic component may be a pharmacologically inert compound that promotes the solidification process normally provided by a hydrophobic drug.

For purposes of therapy, a polymeric drug delivery system is administered to a subject in a therapeutically effective amount. A polymeric composition is said to be administered in a “therapeutically effective amount” if the amount administered is physiologically significant. An agent is physiologically significant if its presence results in a detectable change in the physiology of a recipient subject.

The polymeric compositions described herein may be used to treat a variety of animals. In particular, the polymeric compositions are useful for the treatment of mammals, including humans. Various uses of the polymeric compositions, including the drug delivery systems, for human therapy are described above. However, the drug delivery system can also be used for veterinary applications, such as for the treatment of tumors in either farm or domestic animals. In addition, the drug delivery system is useful for the treatment of arthritis, since this disease is common in many animals (e.g., dogs), and arthritis noticed by animal owners due to the visible interference of normal gait in arthritic animals. The drug delivery system may also be useful in the veterinary treatment of restenosis or post-surgical adhesions. In general, the choice of drugs for veterinary applications would be the same as the examples described given for human therapy.

The present invention, thus generally described, will be understood more readily by reference to the following examples, which are provided by way of illustration and are not intended to be limiting of the present invention.

### EXAMPLES

In the Examples that follow, DL-lactide and glycolide were purchased from PURAC America (Lincolnshire, Ill.; http://www.purac.com). ε-Caprolactone and stannous octoate were purchased from Aldrich and Sigma Chemicals (each in Milwaukee, Wis.), respectively. Poly(ethylene glycols), (PEGs) with number average molecular weights between 200 and 8,000 were purchased from Union Carbide Corp. (Danbury, Conn.; http://www.unioncarbide.com). All other reactants and reagents were obtained from established supply houses, e.g., Sigma-Aldrich (Milwaukee, Wis., http://www.aldrich.sial.com), Fisher Scientific Co. (Hampton, N.H.; http://www.fisher1.com),

The following abbreviations, as used herein, are defined as follows: CL α-caprolactone); DLLA (DL-lactide); DSC (Differential Scanning Calorimetry); g (gram, grams); GPC (gel permeation chromatography); NMR (nuclear magnetic resonance); PCL (poly(ε-caprolactone); PDLLA (poly-DL-lactide); PE (polyester); PEG (polyethylene glycol); PGA (polyglycolide); PLA (polylactide); PLC (poly(DL-lactide-co-α-caprolactone); PLGA (poly(lactide-co-glycolide); PTFE (poly(tetrafluoroethylene), and TB (triblock, triblock copolymer); and T_{g}, (glass transition temperature).

### Example 1

### Synthesis and Characterization of Triblock Copolymers

(a) Synthesis

Triblock (TB) copolymers were synthesized through ring opening polymerization using the monomers DL-lactide, glycolide, ε-caprolactone and PEG. Stannous octoate was used as catalyst.

i. Small Scale Reaction

TB copolymers were synthesized on a small scale by transferring a total of 20 g reactive monomers, with the desired weight ratios of the reactants, into a 50 mL glass ampoule. Stannous octoate (0.1 mL) was added to the ampoule. The ampoule was connected to a vacuum pump and sealed using a propane MicroTorch. The sealed ampoule was then immersed in a 140° C. mineral oil bath. The oil bath was heated by a hot plate connected to a temperature controller. Immediately after the monomers melted, the ampoule was taken out, vortex mixed, and then put back into the oil bath. The ampoule was maintained in the oil bath at elevated temperature for 3-4 hours. To stop the polymerization, the ampoule was opened and the polymer was poured into a 20 mL glass scintillation vial.

ii. Large Scale Laboratory Reaction

The synthesis of the TB copolymers has been scaled up to 400 g batch size. A total of 400 g of reactive monomers, having the desired weight ratios, were weighed into a 500 mL one-neck round-bottomed flask. A 2-inch Teflon coated stirring bar was added to the flask. A glass stopper was used to seal the flask. The flask was then immersed, up to the neck, into a pre-heated oil bath (140° C.). The oil bath was heated by a hot plate connected to a temperature controller. After the monomers melted and the temperature reached 140° C., 2 mL of stannous octoate (catalyst) was added to the flask. The flask was shaken immediately after the addition of the stannous octoate to ensure fast mixing. The flask was then returned to the oil bath. The reaction was allowed to proceed for 6 hours after the addition of the catalyst. Then the polymer melt was poured into a glass jar, capped and stored at 2-8° C.

(b) Characterization

The chemical compositions of the TB copolymers may be determined by ¹H-NMR. To conduct ¹H-NMR analysis, a polymer sample is dissolved in CDCl₃ at 1-10% w/v concentration, and 0.5 mL of the solution is placed into an NMR tube. The ¹H-NMR spectrum is obtained using a Bruker AC-200E NMR instrument (200 MHz) (Bruker Instruments Inc., Billerica, Mass.; http://www.bruker.com).

The polymer molecular weight and its distribution were determined at 40° C. by GPC using a 1100 Series HPLC system (Hewlett-Packard, Palo Alto, Calif.; http://www.hp.com) using a mix-D HLPC Gel column and a refractive index detector. The mobile phase was chloroform with a flow rate of 1 mL/min. The copolymer was dissolved in chloroform at a concentration of 0.2-0.5% w/v. The injection volume was 50 μL. The polymer molecular weight was determined relative to polystyrene standards. The data was collected and processed using PL GPC 3.02 software purchased from Polymer Laboratories, Inc. (Amherst, Mass.; http://www.polymerlabs.com).

The thermal properties of the copolymers were analyzed using a Perkin Elmer Pyris 1 DSC (The Perkin-Elmer Corp., Norwalk, Conn.; http://www2.perkin-elmer.com). The copolymer, or paclitaxel/copolymer, was weighed (3-5 mg) into a crimped open aluminum sample pan, which was placed on the DSC sample holder. The heating rate was 40° C./min, starting at −100° C. and continuing to 100° C.

Twenty TB copolymers were synthesized and characterized, as summarized in TABLES 1 and 2. Thirteen of the TB copolymers were prepared and characterized as summarized in TABLE 1. In the first column of TABLE 1, titled PLC-PEG-PLC, the reactive monomers used to prepare each TB copolymer are summarized, using the nomenclature M-X/Y/Z. According to this nomenclature, X, Y and Z refer to the weight percentage of DL-lactic acid, ε-caprolactone and PEG, respectively, used in preparing the TB copolymer, where these percentage values are based on the total weight of the three monomers. In TABLE 2, the first column is designated PDLLA-PEG-PDLLA, and uses the nomenclature M-X/Y to identify the reactants, and relative amounts thereof, used to prepare each of seven TB copolymers. In connection with TABLE 2, X and Y are the weight percentages of DL-lactide and PEG used to prepare the TB copolymer, where these weight percentages are based on the total weight of the reactants. In each of TABLES 1 and 2, the designation M represents the molecular weight of the PEG, as reported by the supplier (Union Carbide).

The polymer molecular weights, physical states and thermal properties for the 20 TB copolymer are set forth in TABLES 1 and 2. The molecular weight values obtained by GPC measurements (see column titled “M.W.”, and subtitled “GPC”) correlated linearly to the calculated molecular weight (see column titled “M.W.”, and substitled “Cal.”). In TABLE 1, the molecular weights were calculated according to the equation M.W. (Cal.)=(1+(W_{DLLA}+W_{CL})/W_{PEG})×MW_{PEG}, where W represents monomer weight. In TABLE 2, the molecular weights were calculated according to the equation M.W. (Cal.)=(1+W_{DLLA}/W_{PEG})×MW_{PEG}, where W represents monomer weight.

A comparison of M.W. (Cal.) and M.W. (GPC) demonstrates that polymer molecular weight can be controlled and predicted based on the starting raw materials. It is also seen that the polymer molecular weight increases with both increasing PEG molecular weight and with increasing weight percentage of the polyester monomer, as predicted in the calculating equations.

At a constant DLLA/CL/PEG ratio of 35/35/30, polymer viscosity (rheology) increased from liquid to pasty with increasing PEG molecular weight. The melting temperature Tₘ, and melting enthalpy ΔHₘ, both increased with increasing PEG molecular weight (see TABLE 1). At constant PEG molecular weight (3350) and percentage (30%), the PLC-PEG-PLC changed from a paste to a solid with increasing DLLA content. PDLLA-PEG-PDLLA is a harder material than PLC-PEG-PLC (TABLES 1 and 2) since the glass transition temperature of PLC (about 0° C., 50:50 DLLA:CL) is lower than that of PDLLA (55° C.).

In TABLES 1 and 2, “ND” stands for “not determined”.

TABLE 1
PROPERTIES OF SELECTED PLC-PEG-PLC COPOLYMERS

| | | | | | Thermal | | |
|---|---|---|---|---|---|---|---|
| | M.W. | | Physical State | | Properties (DSC) | | |
| PLC-PEG-PLC | Cal. | GPC | Ambient | 37° C. | T_{g}, ° C. | Tₘ, ° C. | ΔHₘ, J/g |
| 200-35/35/30 | 667 | 1372 | Flowable | Flowable | ND³ | ND | ND |
| 600-35/35/30 | 2000 | 4173 | Flowable | Flowable | ND | ND | ND |
| 1000-35/35/30 | 3333 | 5542 | Flowable | Flowable | ND | ND | ND |
| 2000-35/35/30 | 6667 | 9811 | Paste | Flowable | −50.0 | 16.0 | −13.2 |
| 3350-25/25/50 | 6700 | 9378 | Paste | Paste | ND | ND | ND |
| 3350-30/30/40 | 5025 | 11771 | Paste | Paste | ND | ND | ND |
| 3350-35/35/30 | 11167 | 14584 | Paste | Paste | −49.3 | 26.7 | −21.8 |
| 3350-50/20/30 | 11167 | ND | Semisolid | Paste | ND | ND | ND |
| 3350-60/10/30 | 11167 | ND | Solid | Paste | ND | ND | ND |
| 3350-70/0/30 | 11167 | ND | Solid | Paste | ND | ND | ND |
| 4000-35/35/30 | 13333 | 14694 | Paste | Paste | −46.8 | 31.3 | −25.1 |
| 4600-35/35/30 | 15333 | 19848 | Paste | Paste | −41.7 | 34.0 | −29.6 |
| 8000-35/35/30 | 26667 | 34226 | Paste | Paste | −53.3 | 38.7 | −30.8 |

TABLE 2
PROPERTIES OF SELECTED PDLLA-PEG-PDLLA COPOLYMERS

| | M.W. | | Physical State | |
|---|---|---|---|---|
| PDLLA-PEG-PDLLA | Cal. | GPC | Ambient | 37° C. |
| 200-70/30 | 667 | 1131 | Flowable | Flowable |
| 600-70/30 | 2000 | 3124 | Paste | Flowable |
| 1000-70/30 | 3333 | 4970 | Paste | Paste |
| 2000-70/30 | 6667 | 10439 | Semisolid | Paste |
| 3350-70/30 | 11167 | 16950 | Solid | Semisolid |
| 4600-70/30 | 15333 | 23197 | Solid | Solid |
| 8000-70/30 | 26667 | 42962 | Solid | Solid |

### Example 2

### Synthesis and Characterization of Paclitaxel/TB Copolymer Paste and Paint Formulations

(a) Synthesis

Paclitaxel was purchased from Hauser, Inc. (Boulder, Colo.), and DCM (dichloromethane) was from Fisher Scientific Co. (Hampton, N.H.). TB copolymers were synthesized as described in EXAMPLE 1. Paclitaxel pastes, suitable, for example, for the treatment of cancer, were made from PLC-PEG-PLC 4000-35/35/30. Paclitaxel paints, suitable, for example, for the prevention of post-surgical adhesion, were made from PLC-PEG-PLC 2000-35/35/30.

A TB copolymer was dissolved in DCM at an accurately known concentration (in the range of 10-15% w/w). The polymer solution was centrifuged at 3000 rpm for 0.5 hr and the supernatant was divided into glass beakers and weighed. A paclitaxel DCM stock solution with an accurately known concentration (in the range of 10-20 mg/mL) was prepared using a volumetric flask. Based on the amount of the polymer, the volume of the paclitaxel DCM solution needed for a certain drug loading was calculated, and the paclitaxel solution was transferred to the beakers containing the polymer supernatant. The beakers were magnetically stirred in a fume hood for at least 16 hours to provide for solvent evaporation. The remaining solvent was removed by vacuum drying at −100 kPa and 50-55° C. for at least 8 hours. The paste or paint was then drawn into plastic syringes and sterilized by 2.5 Mrad γ-ray irradiation. The syringes were stored at 2-8° C. in a refrigerator.

(b) Characterization

The formulations prepared in (a) above were characterized using ¹H-NMR, GPC and DSC, according to the protocols described in EXAMPLE 1. Paclitaxel content and stability was determined with a HPLC system (1100 Series, Hewlett-Packard) using a Curosil PFP column (5 μm, 250×4.60 mm, Phenomenex, Torrance, Calif.) and an ultraviolet spectrophotometric detector set at 227 nm. The temperature of the column oven was 28° C. The injection volume was 20 μl and the flow rate was 2 mL/min. The mobile phase was a gradient system of acetonitrile and water combined as set forth in TABLE 3.

TABLE 3
HPLC MOBILE PHASE

| Time, min | Acetonitrile, % | Water, % |
|---|---|---|
| 0 | 37 | 63 |
| 40 | 37 | 63 |
| 55 | 60 | 40 |
| 55.5 | 100 | 0 |
| 65 | 100 | 0 |
| 68 | 37 | 63 |
| 78 | 37 | 63 |

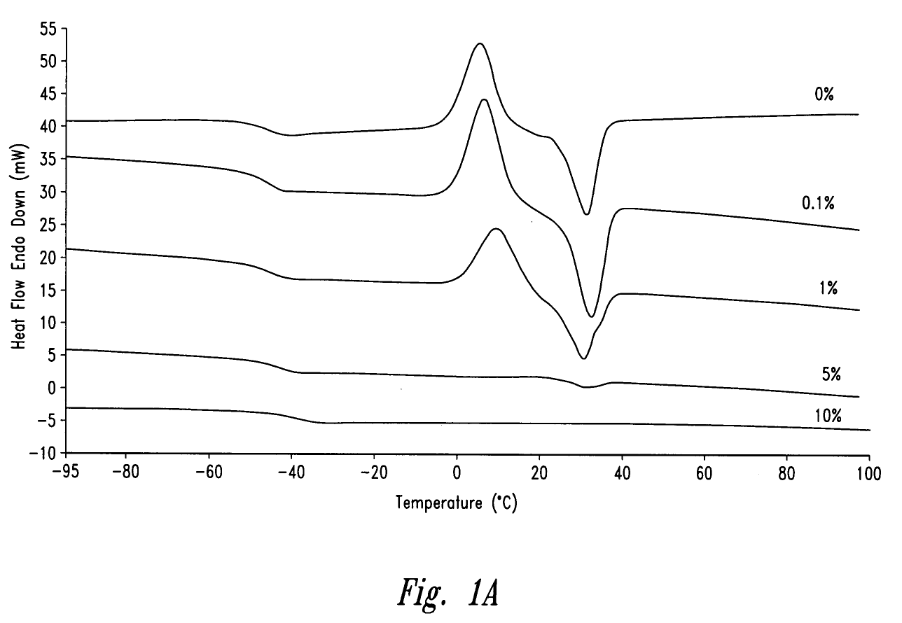

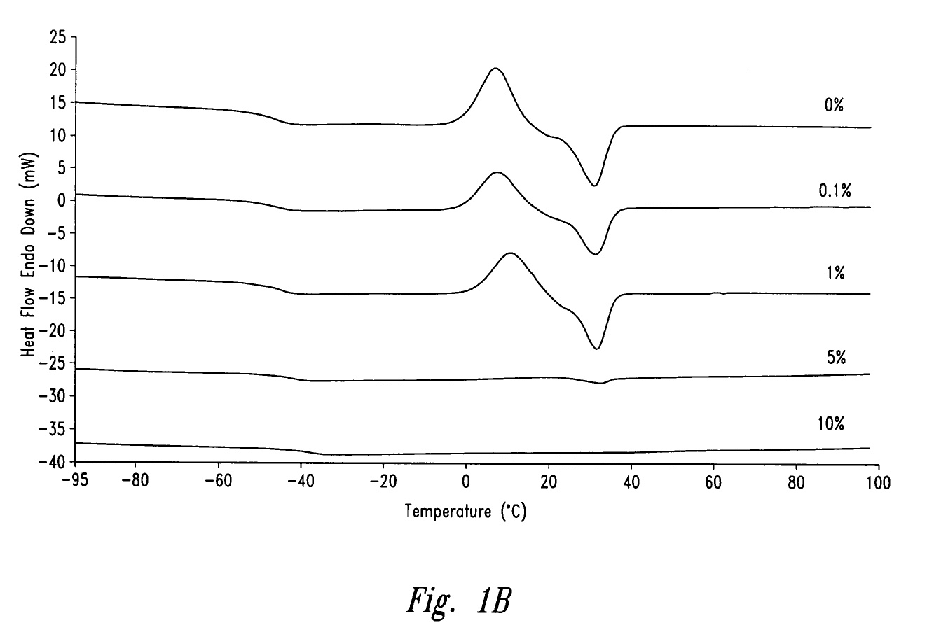

The results from various physicochemical characterizations of paclitaxel/PLC-PEG-PLC 4000-35/35/30 pastes are shown in TABLE 4 and FIGS. 1A and 1B. Paclitaxel content as measured by HPLC was seen to correlate with the targeted loading. In both cases of prior and after γ-ray irradiation, no paclitaxel degradation occurred as revealed by HPLC (data not shown). The polymer molecular weights measured by GPC before and after the irradiation, regardless of paclitaxel loading, were 15234±498 and 14628±443, respectively. This showed that the average polymer molecular weight decreased by 4.0% after the irradiation but the decrease was not statistically significant (p<0.076 two tail). ¹H-NMR showed that there was no chemical composition change after the irradiation.

FIGS. 1A and 1B show DSC thermograms of PLC-PEG-PLC 4000-35/35/30 with various paclitaxel loadings. FIG. 1A is before γ-ray irradiationm, while FIG. 1B is after 2.5 Mrad γ-ray irradiation. Both Tg and melting enthalpy (ΔHₘ) decreased with paclitaxel loading while polymer melting temperature (Tₘ) was unchanged (FIGS. 1A and 1B). No paclitaxel melting was observed when DSC measurements were conducted up to 250° C. (data not shown). It therefore can be concluded that the irradiation did not have an effect on the thermal properties of the paste formulations.

TABLE 4
PHYSICOCHEMICAL CHARACTERISTICS
OF PLC-PEG-PLC 4000-35/35/30 PASTES

| | | | | Thermal | | |
|---|---|---|---|---|---|---|
| Paclitaxel | | Drug | | Properties (DSC) | | |
| Loading | γ-ray | content (HPLC) | Polymer MW (GPC) | T_{g}, ° C. | Tₘ, ° C. | ΔHₘ, J/g |
| 0% | No | 0% | 15998 | −47.3 | 30.7 | −24.9 |
| 0.1% | No | 0.09% | 15154 | −47.0 | 32.0 | −25.3 |
| 1% | No | 0.96% | 14602 | −46.4 | 30.0 | −22.4 |
| 5% | No | 4.45% | 15219 | −44.3 | 30.7 | −1.5 |
| 10% | No | 9.26% | 15197 | −40.5 | None | 0 |
| 0% | Yes | 0% | 14664 | −47.8 | 30.0 | −24.0 |
| 0.1% | Yes | 0.09% | 15024 | −47.2 | 30.0 | −23.5 |
| 1% | Yes | 0.9% | 14812 | −47.1 | 30.7 | −21.6 |
| 5% | Yes | 4.47% | 14769 | −43.5 | 32.0 | −1.6 |
| 10% | Yes | 8.89% | 13871 | −39.5 | None | 0 |

Selected physicochemical characterizations of paclitaxel/PLC-PEG-PLC 2000-35/35/30 paints are shown in TABLE 5. Paclitaxel content measured by HPLC correlated with the targeted loading. In both cases of prior and after γ-ray irradiation, no paclitaxel degradation occurred. The polymer molecular weights before and after the irradiation, regardless of paclitaxel loading, were 10663±30 and 10818±23, respectively. This indicated that the irradiation did not affect polymer molecular weight.

TABLE 5
PHYSICOCHEMICAL CHARACTERISTICS
OF PLC-PEG-PLC 2000-35/35/30 PAINTS

| Paclitaxel | | | |
|---|---|---|---|
| Loading | γ-ray irradiation | Drug content, HPLC | Polymer MW, GPC |
| 0% | No | 0% | 10631 |
| 0.1% | No | 0.1% | 10689 |
| 1% | No | 0.89% | 10670 |
| 0% | Yes | 0% | 10805 |
| 0.1% | Yes | 0.1% | 10844 |
| 1% | Yes | 0.89% | 10805 |

### Example 3

### Release of Paclitaxel from PLC-PEG-PLC Paste

(a) Procedure

HPLC grade acetonitrile and water were purchased from Calcdon Laboratories (Georgetown, Ontario, CANADA). Phosphates were purchased from BDH Inc. (Toronto, Ontario, CANADA; http://www.bdhinc.com). Albumin Fraktion V was bought from Boehringer Mannheim, Germany (now part of F. Hoffmann-La Roche Ltd., Basel, SWITZERLAND, http://www.roche.com).

Paclitaxel loaded PLC-PEG-PLC formulations were weighed (13-17 mg of paste, or 50-100 mg paint) into 14 mL glass test tubes containing 10 mL 0.02 M phosphate buffered saline with 0.8 g/L albumin (in PBSA, pH 7.4). The PBSA solution was made by dissolving 0.32 g sodium dihydrogen orthophosphate (NaH₂PO₄.H₂O), 2.60 g sodium phosphate monohydrate (Na₂HPO₄), 8.22 g sodium chloride and 0.8 g albumin in 1 L distilled water. The test tube was sealed with a PTFE lined screw cap (Glas-Col, Terre Haute, Ind., http://www.glascol.com) and tumbled at about 50 rpm in a 37° C. oven. Periodically, the tube was centrifuged (J6-HC centrifuge, Beckman) at 2,000 rpm for 0.5 hr. The supernatant was withdrawn and replaced with fresh PBS albumin (PBSA) buffer.

To analyze the paclitaxel concentration in the release medium, 0.5 mL supernatant was mixed with 0.5 mL acetonitrile and centrifuged at 10,000 rpm for 5 minutes using a bench top centrifuge (Micromax, IEC International, Needham, Mass.). The amount of paclitaxel in this supernatant was then analyzed using a HPLC system (1100 Series, Hewlett Packard). HPLC analysis was performed using a ODS Hypersil column (5 μm, 125×4 mm, Hewlett Packard) and an ultraviolet spectrophotometric detector set at 232 nm. The temperature of the column oven was 28° C. The injection volume was 5 μL. The mobile phase was 45% acetonitrile and 55% water, and the flow rate was 1 mL/min.

(b) Results

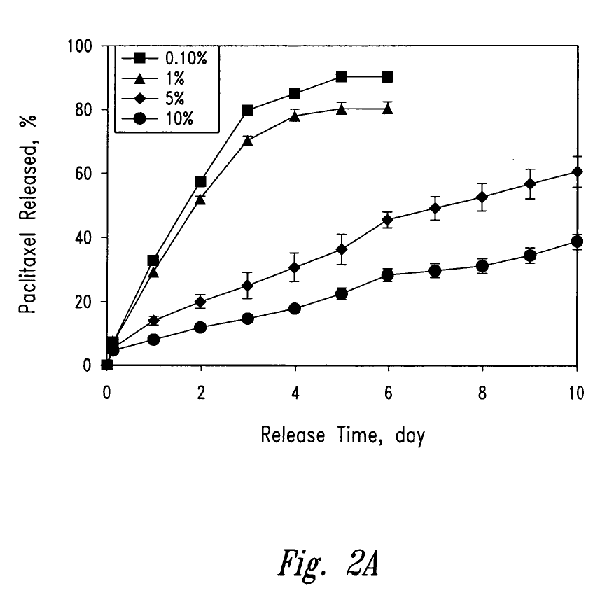

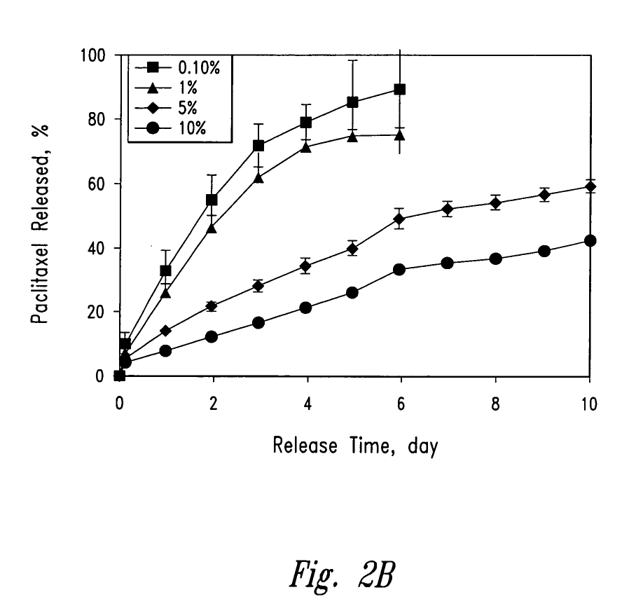

The releases of paclitaxel from PLC-PEG-PLC 4000-35/35/30 pastes into 37° C. PBSA are shown in FIGS. 2A and 2B. FIG. 2A is a graph representing the release of paclitaxel from non-irradiated sample, while FIG. 2B is a graph representing the release of paclitaxes from a sample irradiated with 2.5 Mrad γ-ray, where the samples are PLC-PEG-PLC 4000-35/35/30 pastes in PBSA at 37° C.

The release profiles depended on paclitaxel loading but were not affected by the γ-ray irradiation. Similar release profiles were observed between low drug loadings of 0.1% and 1% w/w, where paclitaxel was released rapidly in the first three days (about 70%), then slowly in the next two days (about 10%) and finally diminished after 5 days. At high drug loadings of 5% and 10%, the released paclitaxel increased almost linearly with time. About 60% and 40% of paclitaxel was released from the 5% and 10% loaded pastes within 10 days, respectively. The pastes broke into small pieces during the course of the release study. Both the number and the size of the broken pieces of the pastes were reduced due to polymer degradation (see EXAMPLE 4) and paclitaxel release. No paclitaxel crystals were observed in the cases of 0.1% and 1% loaded samples. On the other hand, needle shaped paclitaxel crystals were seen under an optical microscope after 2 days release in the cases of 5% and 10% drug loaded samples. The formation of paclitaxel crystals explained the slow release from the 5% and 10% pastes. Another reason for fast release of the low loading samples may be that that the degradation products of the triblock copolymers (for example, amphiphilic PLC-PEG) have a solubilization effect on paclitaxel, which is more pronounced at lower paclitaxel loading.

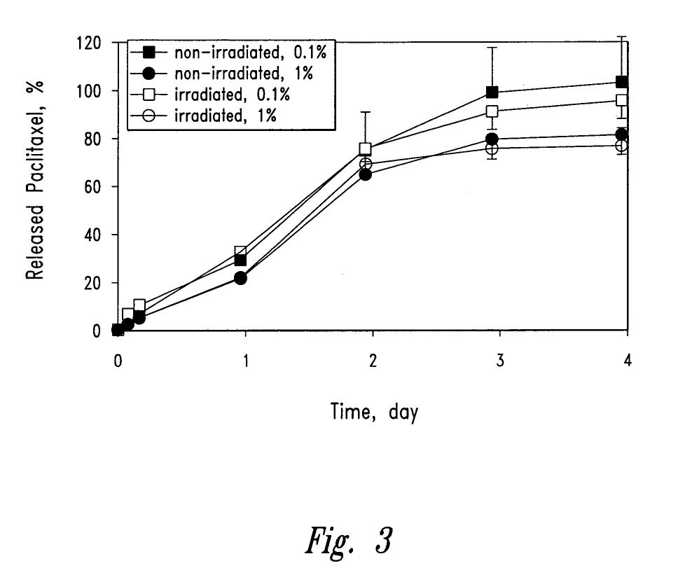

The releases of paclitaxel from PLC-PEG-PLC 2000-35/35/30 paints (both irradiated and non-irradiated samples) into 37° C. PBSA are shown in FIG. 3. The release profiles were not affected by the γ-ray irradiation and were slightly depended on paclitaxel loading. Similar release profiles were obtained for all the samples, where paclitaxel was released rapidly in three days (80-100% release). The paints broke up into small pieces during the release and almost diminished after 4 days release. No paclitaxel crystals were observed.

### Example 4

### Degradation of PLC-PEG-PLC

(a) Procedure

A polymer degradation study of the pastes and paints in PBSA was done in the same manner as in the release study (see EXAMPLE 3) except that paclitaxel concentration in the release medium was not analyzed. The degradation of pure PLC-PEG-PLC 3350-35/35/30 and a blend of PLC-PEG-PLC 4600-35/35/30 and MePEG 350 was carried out without buffer change. At different times, the samples were washed three times with distilled water, followed by centrifuging and decanting and then dried in a vacuum oven (−100 kPa) for three days. The dry weight, paclitaxel content and polymer molecular were determined using an analytical balance (Sartorius BP210D, Edgewood, N.Y.), HPLC and GPC. The HPLC samples were prepared by adding acetonitrile and then an equal volume of water to the dried residue followed by vortexing, sonication, and centrifugation to obtain supernatant.

(b) Results

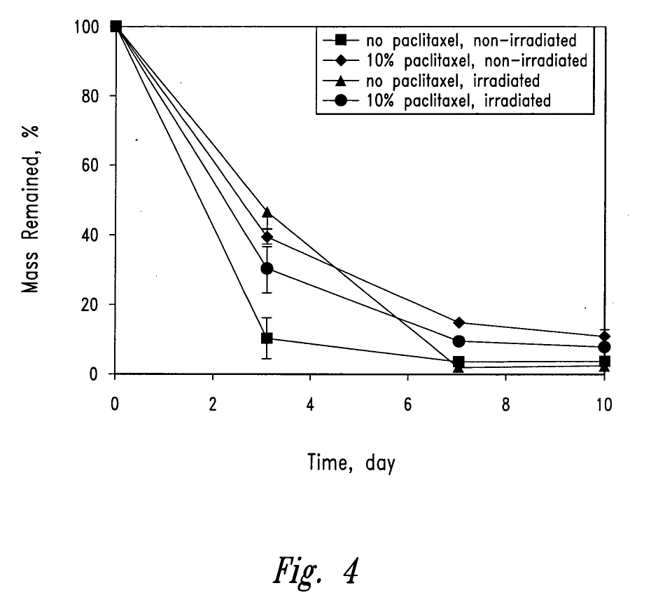

The dry mass loss of PLC-PEG-PLC 4000-35/35/30 is shown in FIG. 4 (sample size 14-18 mg). It can be seen that the polymer degraded in 7 days. The irradiation did not have an significant effect on the rate of mass loss. The 10% paclitaxel loaded pastes had a slower mass loss rate. Analysis of residual paclitaxel using HPLC showed that the drug percentage was as high as 42% in the remaining mass. After 3 days degradation, polymer molecular weights decreased by 14.9+8.4%, regardless of paclitaxel loading and γ-ray irradiation. The polymer molecular weight was not detectable using GPC after 7 days and 10 days degradation. The dry mass loss on day 3 was 63.8±18.5%, which was more significant than the polymer molecular weight loss. This tends to indicate that the polymer degraded through a surface erosion mechanism.

The degradation of the PLC-PEG-PLC 2000-35/35/30 paints after 4 days in 37° C. PBSA is shown in TABLE 6. Within 4 days, about 90% of total mass was lost and paclitaxel was almost totally released. The γ-ray irradiation did not have a significant effect on polymer degradation rate. The polymer molecule weight was not detectable by GPC after 4 days since the amount of residual sample was so small. The sample size subjected to degradation was on the order of 50-100 mg.

TABLE 6
THE DEGRADATION OF PLC-PEG-PLC 2000-35/35/30 PAINTS
AFTER FOUR DAYS IN 37° C. PBSA

| Paclitaxel | | | Paclitaxel |
|---|---|---|---|
| Loading | γ-ray irradiation | Mass remaining, % | remaining, % |
| 0% | No | 7.6 ± 1.6 | None |
| 0.1% | No | 10.0 ± 3.1 | 0.18 |
| 1% | No | 11.4 ± 1.6 | 0.14 |
| 0% | Yes | 9.9 ± 2.9 | None |
| 0.1% | Yes | 10.0 ± 0.9 | 0.47 |
| 1% | Yes | 10.6 ± 0.5 | 0.18 |

The degradation of PLC-PEG-PLC 3350-35/35/30 with larger sample size (200 mg) was studied and the results are shown in TABLE 7. The polymer weight loss was 62% in 4 weeks and the pH declined slightly due to the acidic degradation products generated. At the same time, polymer molecular weight decreased by only 20.6%. No significant change in thermal properties (DSC) and in chemical compositions (¹H-NMR) occurred. Again, this tends to indicate that surface erosion occurred in this case.

TABLE 7
DEGRADATION OF PLC-PEG-PLC 3350-35/35/30

| | | | | Thermal | | |
|---|---|---|---|---|---|---|
| Time, | | | | Properties, DSC | | |
| day | Mass loss, % | MW | pH | T_{g}, ° C. | Tₘ, ° C. | ΔHₘ, J/g |
| 0 | 0 | 16420 | 7.30 | −49.3 | 31.8 | −36.1 |
| 6 | 18.64 ± 0.27 | 14860 | 6.60 | −46.2 | 32.8 | −28.0 |
| 10 | 27.42 ± 0.13 | 14970 | 6.58 | −45.4 | 33.1 | −29.4 |
| 23 | 53.68 ± 3.79 | 13929 | 6.00 | −44.5 | 33.8 | −22.6 |
| 28 | 62.27 ± 1.10 | 13032 | 6.20 | −44.3 | 33.8 | −22.6 |

The degradation of a blend of 90% PLC-PEG-PLC 4600-35/35/30 and 10% MePEG 350 with 200 mg sample sizes was also studied, and the results are shown in TABLE 8. The polymer weight loss was 96% in 41 days and the pH declined slightly due to the acidic compounds generated in the degradation. At the same time, polymer molecular weight remained as high as 12,000. This supports the view that the triblock copolymer degrades through a surface erosion mechanism.

TABLE 8
DEGRADATION OF BLEND OF 90% PLC-PEG-PLC 4600-35/35/30
AND 10% MEPEG 350

| Time, day | Mass loss, % | MW | pH |
|---|---|---|---|
| 0 | 0 | ND | 7.41 |
| 7 | 39.59 ± 3.33 | 16981 | 6.62 |
| 14 | 57.38 ± 0.95 | 15537 | 6.29 |
| 22 | 74.97 ± 1.92 | 14268 | 6.14 |
| 28 | 87.23 ± 2.30 | 13473 | 5.95 |
| 41 | 96.05 ± 0.95 | 11945 | 5.30 |

### Example 5

### Manufacture of Drug-Loaded TB:MePEG Compositions

Paclitaxel was obtained from Hauser chemical company, Boulder Colo. Amphoterecin, Nystatin, and Tretinoin were a kind gift from Dr. K. Wasan (University of B.C., Vancouver, B.C., Canada). All other hydrophobic drugs, including curcumin, genistein, tretinoin, nystatin, amphoterecin, and camptothecin were obtained from Sigma Chemicals (St. Louis, Mo.). Methotrexate and colchicine (Sigma Chemicals) were used as examples of non-hydrophobic drugs.

Compositions were manufactured by combining TB copolymer, MePEG 350 (molecular weight 350, Union Carbide, Danbury, Conn.) and drug, and then warming the three components to 50° C. in specified ratios in a 20 mL glass scintillation vial (Fisher Scientific) and levigating the mixture for 5 minutes to form a solution or homogenous suspension of drug in polymer.

The manufacture of 2 gram batches of 10% (w/w) loaded paste was achieved by blending 200 mg of paclitaxel (Hauser Chemical Co. Boulder, Colo.) into 1080 mg of MePEG 350 (Union Carbide, Danbury, Conn.) for 5 minutes at 40° C., followed by 720 mg of a TB copolymer (4600-35/35/30). This mixture was stirred for 15 minutes at 50° C. such that all the paclitaxel was dissolved in the liquid polymer blend and then the polymer-drug solution was drawn up into 1 mL luer lock syringes (BD labware, Bedford, Mass.) and stored at 4° C. until use. Control paste (no drug) was manufactured by blending TB 4600-35/35/30 with MePEG in a 40:60 ratio (w/w) at 50° C. for 5 minutes, followed by syringe capture and storage at 4° C. until use.

To assess the stability of the compositions, the formulations were cooled and stored at −20° C. for 24 hours and then allowed to warm up to room temperature for two hours. The compositions were then observed for evidence of drug crystallization.

All concentrations of paclitaxel (2.5%, 5%, 10%, and 15%) in all blends of TB:MePEG350 (30:70 to 90:10) formed solutions of the drug in the molten polymer matrix during blending at 50° C. Also, when the blends were allowed to cool to room temperature, no evidence of paclitaxel crystallization could be seen using optical microscopy. However, when the paste compositions were allowed to cool to −20° C. overnight and then allowed to warm up to room temperature for two hours, there was evidence of paclitaxel crystals in some compositions. The compositions that had crystals present in the paste after −20° C. storage are presented in TABLE 9. Compositions that had low concentrations of paclitaxel or high concentrations of MePEG350 had no evidence of crystal formation after storage at −20° C.

TABLE 9
EFFECT OF STORAGE AT −20° C. ON PACLITAXEL
CRYSTALLIZATION IN POLYMERIC PASTES
HAVING VARIOUS TRIBLOCK:MEPEG RATIOS

| | 30:70 | 40:60 | 50:50 | 60:40 | 70:30 | 80:20 | 90:10 |
|---|---|---|---|---|---|---|---|
| 2.5% paclitaxel | n/c | n/c | n/c | n/c | n/c | n/c | c |
| 5% paclitaxel | n/c | n/c | n/c | n/c | c | c | c |
| 10% paclitaxel | n/c | n/c | n/c | c | c | c | c |
| 15% paclitaxel | n/c | n/c | c | c | c | c | c |

n/c: No crystals present.
c: Crystals present.

Storage at −20° C. does not affect the formulation for most concentrations of paclitaxel. Drug crystals do not appear in any of the 30:70 or 40:60 pastes following −20° C. storage, so they may stored at this temperature and injected through narrow gauge needles without blockage due to crystals. However, storage at this temperature is not required. It should be noted that the presence of drug crystals would not make the polymer-drug formulation impractical to use. However, drug release characteristics might be modified in such a paste as compared to a paste in which the drug remained fully dissolved.

Using normal finger pressure on a disposable one milliliter syringe, the 30:70 and 40:60 (TB:MePEG350) blends of paclitaxel (at all loadings) could be extruded through a 22-gauge needle at room temperature. Therefore, these compositions could be easily injected into a patient, without the need to heat the compositions. The 50:50 (TB:MePEG350) blends could also be extruded through a 22-gauge needle when heated to 37° C., and thus may be used for non-invasive surgical treatments at 37° C. without solidifying. These pastes (30:70 to 50:50) are therefore suitable for use without the need for invasive surgery.

All the hydrophobic drugs tested could be blended at 10% loading into the 40:60 (TB:MEPEG350) composition and subsequently extruded through a 22-gauge needle at room temperature. Thus, this composition would be suitable for non-invasive treatments using any hydrophobic drug or combination of hydrophobic drugs.

All other paste formulations are suitable for injections through wider gauge needles, which may be acceptable, for example, for intramuscular or subcutaneous injections. All pastes may be used on surgically exposed areas where an open syringe is used to extrude the paste at room temperature.

All concentrations of paclitaxel (2.5% to 15%) could be blended and dissolved in all ratio blends of TB and MePEG350 without the need for codissolution in an organic solvent. Therefore, there is no danger of residual organic solvents in the polymer blends. Since an organic solvent is unnecessary, the drug is less likely to precipitate out of the paste under long-term storage since it does not need to be initially dissolved in the polymer by codissolution in a solvent. The 40:60 blend containing paclitaxel at 10% was unaffected by high dose radiation and was still extrudable through a 22-gauge needle at 25° C.

At 10% drug loading, all other drugs formed homogenous dispersions or solutions of the drugs in the molten polymer at 50° C. There was some evidence of undissolved drug crystals in the molten polymer at a 10% w/w drug loading for most of these drugs. However, all compositions were free flowing molten fluids at 50° C. using the 40:60 (TB:MePEG350) polymer blend which could be easily extruded through a 22-gauge needle. There was no evidence of undissolved crystals congealing in the polymer blend and the 10% drug loaded 40:60 (TB:MePEG350) blend was considered a homogenous formulation for these drugs.

### Example 6

### Characterization Of Paclitaxel-Loaded TB:MePEG Compositions Using Differential Scanning Calorimetry (DSC)

DSC was performed using a Perkin Elmer Pyris 1 calorimeter. Approximately 10 mg of 10% paclitaxel-loaded paste (40:60 blend of TB:MEPEG350) was placed in an aluminum DSC pan and scanned from 10° C. to 85° C. at a rate of 10° C. per minute.

Differential Scanning Calorimetry

DSC thermograms were collected for each sample blends and included those from several batch formulations. FIG. 5A shows the average peak melting temperature (Tm) for each blend versus the proportion of MePEG present.

As shown in FIG. 5A, pure TB had a Tm of 40.52° C., the 90:10, 70:30, 50:50 and 30:70 formulations had an average Tm values of 38.90° C., 35.04° C., 32.65° C. and 28.65° C., respectively.

Increasing amounts of MePEG caused the depression of the Tm in a linear fashion, having a slope of −0.167, as shown in FIG. 5B. This linear relationship remains relevant for blends that contain 70% MePEG of less. DSC thermograms were also collected from quenched samples at a heating rate of 40° C./min, given in FIG. 5C. with values provided in TABLE 10.

TABLE 10
THERMAL DATA GENERATED FROM DSC ANALYSIS OF THE
QUENCHED PASTE SAMPLES AND THEIR COMPONENTS
(TB, MEPEG) USING CRIMPED OPEN ALUMINUM
PANS AND A HEATING RATE OF 40° C./MIN.

| | | | ΔH of | | | | |
|---|---|---|---|---|---|---|---|
| | | | crystallization | Tm | | ΔH of melting | |
| TB:MePEG | Tg (° C.) | Tc (° C.) | exotherm (J/g) | (° C.) | | endotherm (J/g) | |
| 100:0 | −44.51 | 1.29 | 45.14^{a} | 40.47 | | −45.69^{a} | |
| 90:10 | −51.74 | −12.91 | 27.06^{a} | 38.16 | | −28.86^{a} | |
| 80:20 | −60.36 | −32.17 | 29.74^{a} | 37.48 | | −25.05^{a} | |
| 70:30 | −67.10 | −42.70 | 37.98^{a} | 34.84 | | −22.00^{a} | |
| 60:40 | −70.98 | −51.94 | 44.38^{b} | 34.17 | | −18.52^{a} | |
| 50:50 | −74.53 | −57.89 | 40.78^{b} | 31.52 | | −15.61^{a} | |
| 40:60 | −74.97 | ø | ø | −0.99 | 30.20 | −36.90^{b} | −12.85^{a} |
| 30:70 | −75.59 | ø | ø | 0.99 | 28.86 | −45.21^{b} | −9.44^{a} |
| 20:80 | ø | ø | ø | 2.32 | 27.72 | −23.82 | −7.84 |
| 10:90 | ø | ø | ø | 0.29 | ø | −24.09 | ø |
| 0:100 | −98.6 | ø | ø | −24.32 | −4.71 | ø | ø |

^{a}Symmetrical peak.
^{b}Asymmetrical peak (shoulder)
^{ø}Not present/Not measurable

Tg was detectable for the blends having 30% or greater of their composition consisting of TB. The TB had a Tg of −44.51° C. The effect of increasing MePEG at a concentration of 10, 20, 30, and 40% decreased the Tg to −51.74, −60.36, −67.10, and −70.98, respectively. The 50, 60, and 70% formulations had a relatively constant Tg of −74.53, −74.97 and −74.53, respectively. The presence of one Tg indicates miscibility of the polymer blends, therefore, blends that had a TB concentration of 30% (w/w) or greater are fully miscible. Blends that consisted of higher MePEG proportions gave no detectable Tg.

Tc peaks are present in those formulations that had at least half of their composition consisting of TB. TB crystallized at 1.29° C. with increasing MePEG concentrations reducing the peak Tc. This is due to the reduced viscosity of the formulations given by the increasing amounts of MePEG in TB (a reduced Tg indicates a reduced viscosity). Short-chained MePEG molecules act as a plastisizer giving the triblock copolymer chains greater mobility.

The crystallization ΔH for the TB was high at 45.14 J/g. Upon addition of 10% MePEG, there was a drop in ΔH to 27.06 J/g. Increasing amounts of MePEG began to increase the ΔH value of the 80:20, 70:30, 60:40, and 50:50 to 29.74, 37.98, 44.38, and 40.78, respectively. This increasing energy required for crystallization is also due to the plastisizing capability of the MePEG.

A single Tm is present in those formulations that had at least half of their composition consisting of TB. The formulations that had higher than a 50% composition of MePEG showed two melting peaks. Both the Tm and melting AH of the second peak in the 40:60, 30:70, and 20:80 correlate to the single melting endotherm of the 50% or higher TB composition blends. The first peak, that begins to appear at a 40:60 blend, correlates to the endotherm of the MePEG. This indicates that the MePEG is switching roles from being the solute to becoming the solvent (as its concentration increases).

All these data clearly show that the water soluble MePEG is miscible with the TB polymer and depresses the melting point of a blend of MePEG with TB so that the TB becomes more fluid at lower temperatures enabling the blend to be injected through narrow gauge needles.

### Example 7

### Assessment of Drug-Dependent Solidification

Compositions containing 10% w/w drugs (methotrexate, colchicine, curcumin, genistein, tretinoin, nystatin, amphoterecin, camptothecin or paclitaxel) were manufactured using a 40:60 (TB:MEPEG350) composition. Fifteen milligrams of each composition were placed in a 20 mL glass scintillation vial and cooled to 4° C. to form uniform solid pellets. Five milliliters of ice cold phosphate-buffered saline (PBS; pH 7.4) containing 0.2% bovine serum albumin (Fraction 4, Boehringer Mannheim) were placed on top of the pellet, and the vial was placed in a stationary position in a 37° C. oven for 1 hour. At this time, a stir bar was placed in the vial and stirring was commenced at 300 rpm until the paste pellet disintegrated.

When control (no drug) paste blends of 40:60 (TB:MEPEG350) were equilibrated in aqueous media at 37° C. for 1 hour and then stirred, the pellet broke up into pieces within 30 seconds, and therefore, failed to solidify in water at 37° C. (FIG. 6). However, paste compositions containing the highly hydrophobic and water insoluble drugs curcumin, genistein, tretinoin, nystatin, amphoterecin, camptothecin or paclitaxel stayed intact for over 4 minutes under stirring, which indicated that these compositions had solidified. The slightly water soluble drugs colchicine and methotrexate paste compositions did not solidify in aqueous media as shown by the short disintegration time under stirring (2.5 minutes and 30 seconds, respectively, as shown in FIG. 2).

Curcumin, genistein, tretinoin, nystatin, amphoterecin, camptothecin, and paclitaxel are described in The Merck Index as being insoluble in water. Studies by the present inventors showed that the water solubility of all these drugs was less than 2 μg/mL, confirming these data. However colchicine and methotrexate were found to have a water solubility of greater than 500 μg/mL in PBS/albumin (pH 7.4), confirming the high degree of solubility of these drugs in water relative to the other drugs used in this study.

Control (no drug) compositions did not solidify at 37° C. in water whereas pastes loaded with hydrophobic drugs did solidify. Therefore, the presence of a hydrophobic drug is necessary for solidification. Pastes that do not solidify in aqueous media may therefore disperse and be unsuitable for long-term drug release in vivo. The unique drug-dependent solidification characteristics are critical for the compositions' injectability at room temperature (25° C.). The composition sets in vivo to an implant without the need for a catalyst to initiate or assist in the solidification process.

### Example 8

### Effect of Paclitaxel and Camptothecin on LNCaP Cell Proliferation In Vitro

LNCap cells are human metastatic prostate adenocarcinoma cells that are used as a model for prostate cancer (see, e.g., Pousette et al., Prostate 31:198, 1997). In one study, LNCaP cells were seeded at concentrations of 2×10³ and 1×10³ cells/well in 96 well plates. After 48 hours, varying concentrations of paclitaxel or camptothecin (25 μl) were added in each culture well and the plates were incubated at 37° C. for 5 days. After incubation, the cells were fixed with 1% glutaraldehyde solution, and stained for 5 minutes with 0.5% crystal violet. The dye was successively eluted with 100 μl of buffer solution and the absorbance was read on a Titertek Multiskan microplate reader using a wavelength of 492 nm absorbance. Cell growth was expressed as a percentage relative to control wells in the absence of the compound (set at 100%).

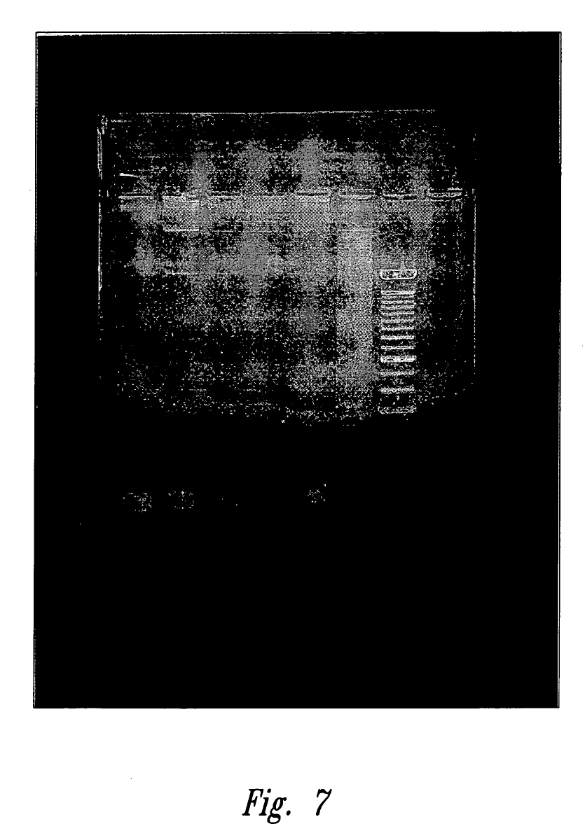

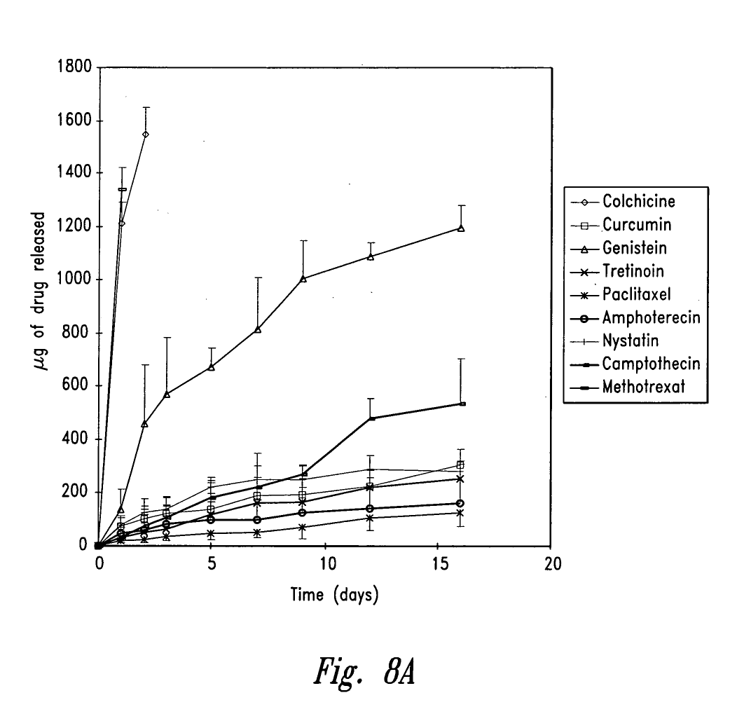

Paclitaxel suppressed LNCaP cell growth in vitro as shown in TABLE 3. Concentrations as low as 0.01 nM caused an inhibition of LNCaP cell growth and the IC₅₀ was approximately 0.09 nM. Apoptosis experiments were performed on the cells in the wells after paclitaxel treatment using DNA fragmentation assays. Briefly, 1×10⁶ LNCaP cells were incubated in 8 cm plates in the presence of medium alone, or with medium containing 0.01 nM, 0.1 nM, 1 nM, 10 nM, or 100 nM paclitaxel. After 18 hours of treatment, genomic DNA was isolated from the cells and analyzed by gel electrophoresis using standard techniques. The results show that treatment with 100 nM paclitaxel induced DNA fragmentation, as evidenced by the laddering effect in sample lane 6 of FIG. 7. In addition, clear evidence of apoptosis was observed by optical microscopy in paclitaxel treated cells as evidenced by the presence of intracellular apoptotic bodies. Therefore, paclitaxel was cytotoxic by an apoptotic mechanism.

TABLE 11
EFFECT OF PACLITAXEL ON LNCAP CELL GROWTH

| N | Paclitaxel (nM) | 492 nm Absorbance | % Growth |
|---|---|---|---|
| 16 | 0.001 | 0.049 ± 0.05 | 100 |
| 16 | 0.01 | 0.40 ± 0.03 | 81 |
| 8 | 0.05 | 0.36 ± 0.02 | 73 |
| 8 | 0.1 | 0.20 ± 0.03 | 40 |
| 8 | 1 | 0.025 ± 0.01 | 5 |
| 8 | 10 | 0.027 ± 0.01 | 5 |
| 8 | 100 | 0.033 ± 0.01 | 6 |

492 nm Absorbance of controls = 0.49 ± 0.06

Camptothecin was extremely potent in its cytotoxic action against LNCaP cells. Concentrations as low as 0.001 nM were toxic to over 60% of cells (TABLE 12). Therefore, the IC₅₀ for this drug against LNCaP cells lies in the femtomolar concentration range.

TABLE 12
EFFECT OF CAMPTOTHECIN ON LNCAP CELL GROWTH

| N | Camptothecin (nM) | 492 nm Absorbance | % Growth |
|---|---|---|---|
| 16 | 0.001 | 0.169 ± 0.05 | 36 |
| 8 | 0.05 | 0.14 ± 0.04 | 29 |
| 8 | 0.1 | 0.10 ± 0.02 | 21 |
| 8 | 1 | 0.10 ± 0.02 | 21 |
| 8 | 10 | 0.088 ± 0.02 | 17 |
| 15 | 100 | 0.038 ± 0.01 | 8 |

492 nm Absorbance of controls = 0.47 ± 0.05

### Example 9

### Determination of In Vitro Drug Release

Formulations containing 10% w/w drugs (methotrexate, colchicine, curcumin, genistein, tretinoin, nystatin, amphoterecin, camptothecin or paclitaxel) were manufactured using a 40:60 (TB:MePEG350) composition. Formulations containing 2.5%, 5%, 10%, and 15% paclitaxel were manufactured using a composition of TB:MePEG350 in ratios from 30:70 to 90:10. Fifteen milligrams of each composition were placed in 20 mL glass scintillation vials and cooled to 4° C. to form uniform solid pellets. Five milliliters of ice cold phosphate-buffered saline (PBS; pH 7.4) containing 0.2% bovine serum albumin (Fraction 4, Boehringer Mannheim) were placed on top of the pellet followed by 5 mL of octanol (Fisher Scientific). The octanol formed an upper imiscible phase on top of the PBS so that any drug released into the PBS would partition into the octanol phase.

The concentration of the drug in the octanol phase was analyzed by either UV/VIS methods or HPLC methods (for paclitaxel only). UV/VIS analysis was performed by determining the absorbance at the specified wavelength. Calibration graphs of the drugs in octanol were established by measuring the absorbance of a set of standards of each drug in octanol in the 0 to 50 μg/mL concentration range. HPLC analysis of paclitaxel was performed using a Waters HPLC system (Mobile phase 58:37:5. ACN:Water:MEOH, 1 mL/min, 20 μl injection, C18 Novapak (Waters) column with detection at 232 nm).

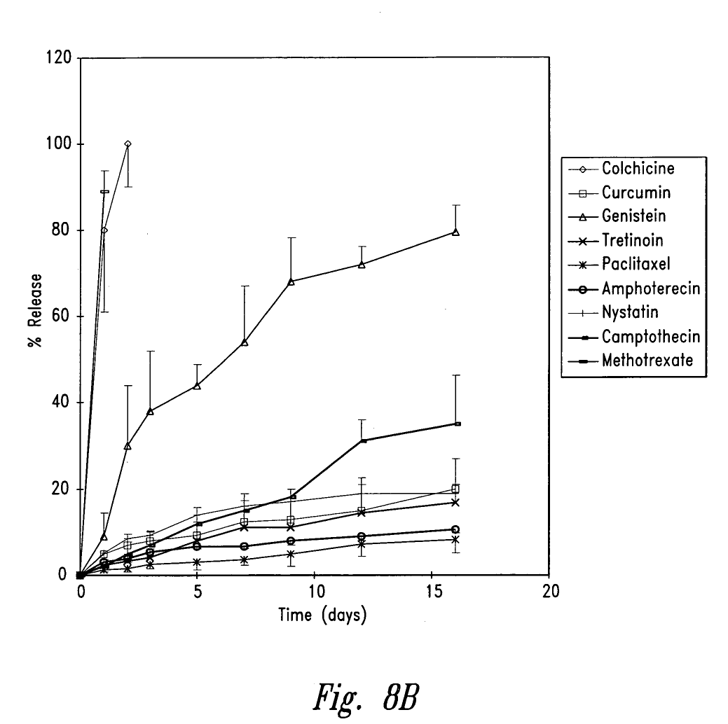

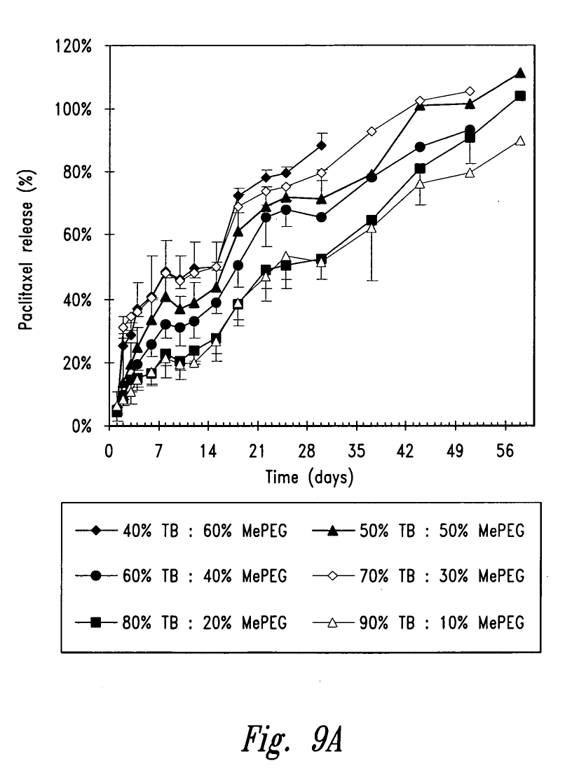

FIGS. 8A and 8B show the drug release profiles for 10% drug-loaded TB:MePEG350 (40:60) paste. The same data is plotted in terms of μg of drug released vs. Time (FIG. 8A) and % of initially loaded drug released vs. Time (FIG. 8B). The moderately water-soluble drugs, methotrexate and colchicine, released almost all the encapsulated drug in the first 24 hours of the drug release experiment. However, all the hydrophobic, water insoluble drugs released the encapsulated drug much more slowly. By day 7, approximately only 10% of the total hydrophobic drug was released. Consistent with the solidification experiments (FIG. 6), which used stir bar agitation, the paste pellets containing the hydrophobic drugs formed distinct solid pellets in PBS which did not to break up in the drug release vials (no agitation). It should be noted that the solidification experiment characterized in FIG. 6 represents an extreme disruption of the paste pellet only one hour after immersion in PBS. The solidification of the drug-polymer blend was observed to be a time dependent process. After one day in the experiment described in this Example (8), all pellets had formed quite solid polymer-drug pellets. On the other hand, the pellets containing the water soluble drugs colchicine and methotrexate had broken up considerably after 2 days (without any agitation).

Genistein, which has a very low water solubility (approximately 1 μg/mL), was released the fastest of all the hydrophobic drugs. However, only 50% of the total amount of encapsulated drug has released from the blend after seven days, indicating that this formulation still represented a slow release formulation of the drug.

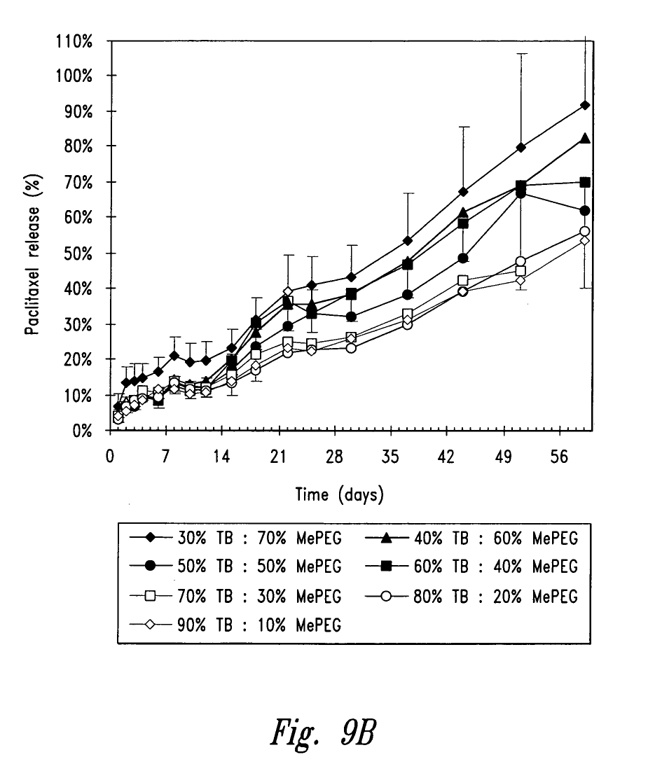

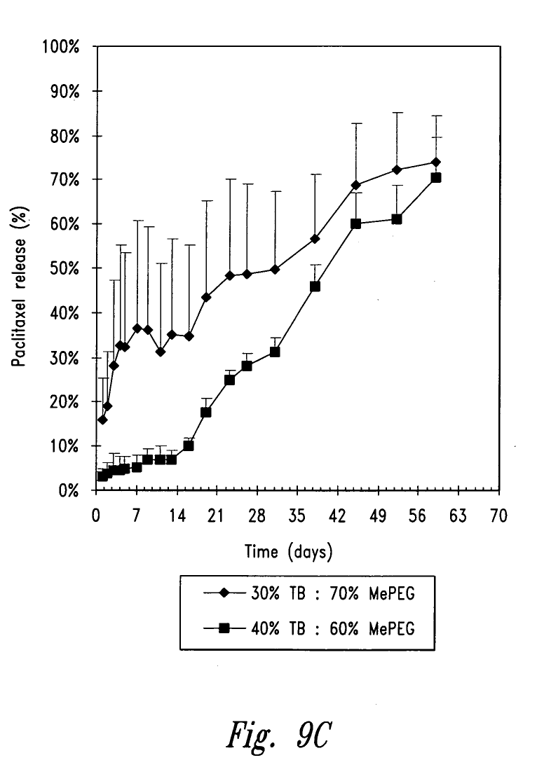

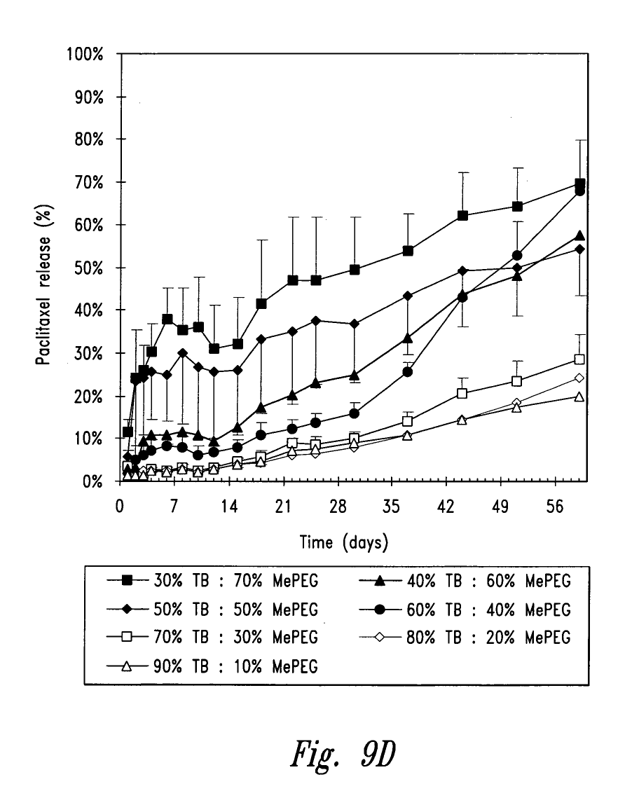

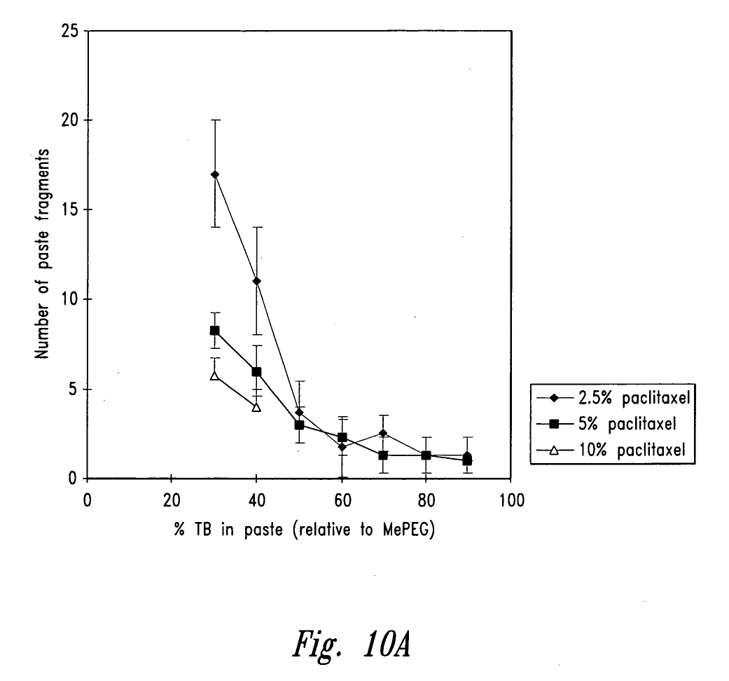

FIGS. 9A, 9B, 9C, and 9D show the release profiles for 2.5% (FIG. 9A), 5% (FIG. 9B), 10% (FIG. 9C) and 15% (FIG. 9D) paclitaxel-loaded pastes composed of TB:MePEG350 blends in the range 30:70 to 90:10. All formulations released paclitaxel with a short burst of drug release in the first three days followed by a slow and steady release profile for the following 40 days. The 2.5% paclitaxel-loaded pastes released the largest percentage of encapsulated drug of all the drug loadings, such that after 30 days between 50% and 100% of encapsulated drug had been released. The 5% paclitaxel-loaded pastes had the next fastest drug release profiles over the whole range of TB:MePEG350 paste blends, such that by day 30, between 23% and 43% of the encapsulated drug had been released. The 10% and 15% paclitaxel-loaded pastes had a wide range of drug release profiles which were dependent on the TB:MePEG ratios as shown in FIGS. 9C and 9D. For both drug loadings (10% and 15%), the 30:70 (TB:MePEG350) blend had a rapid drug release profile, however, there was a large deviation between samples in these groups (n=4). For the 60:40 to 90:10 (TB:MePEG350) blends, the rate of paclitaxel release was low for both 10% and 15% paclitaxel loadings.

Therefore, for all drug loadings (FIGS. 9A, 9B, 9C, and 9D), the rate of drug release was dependent on the percentage of MePEG350 in the blends, such that the 30:70 (TB:MePEG350) blend always gave the fastest drug release profile and the 80:20 and 90:10 blends always gave the slowest drug release profiles.

### Example 10

### Disintegration of Paste Pellets

Pastes containing 2.5%, 5%, and 10% paclitaxel (w/w) were manufactured in TB:MePEG ratios varying from 30:70 to 90:10. Fifteen milligrams of each composition were placed into 20 mL glass scintillation vials and cooled to 4° C. to form uniform solid pellets. Five milliliters of ice cold PBS (pH 7.4) containing 0.2% bovine serum albumin (Fraction 4, Boehringer Mannheim) were placed on top of the pellet followed by 5 mL of octanol (Fisher Scientific). The vials were allowed to sit in a 37° C. oven for 1 month. At 7 days and 30 days, the vials were gently removed from the oven and the degree of disintegration of the original pellet was determined by counting the number of pellet pieces in each vial.

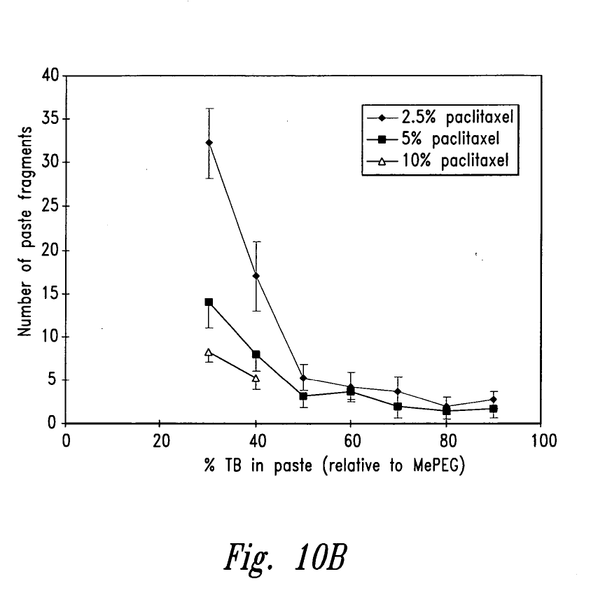

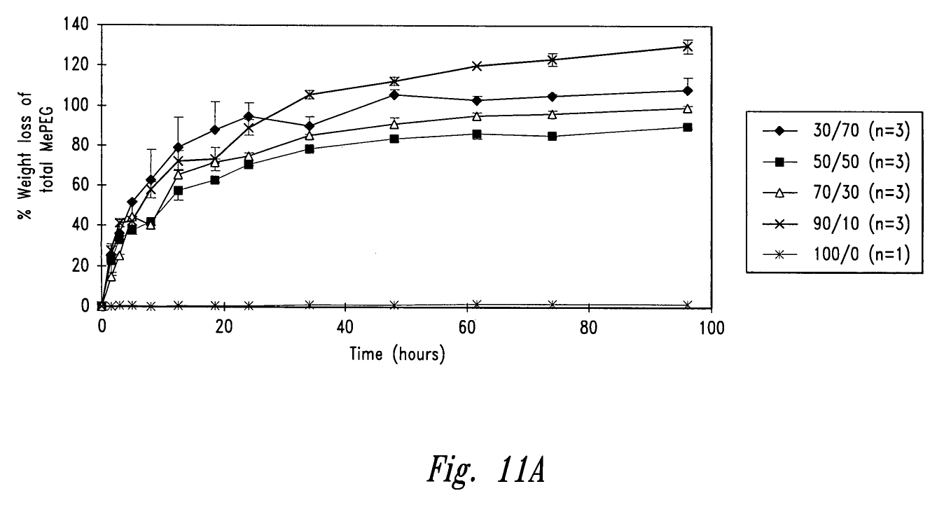

FIGS. 10A and 10B show the disintegration of paclitaxel loaded (2.5%, 5%, and 10% paclitaxel w/w) pastes using 30:70 to 90:10 TB:MePEG350 blends. The data are presented as the total number of paste fragments in PBS/albumin on either day 7 or 30 (37° C.). The 2.5% paclitaxel-loaded 30:70 (TB:MePEG350) paste pellets disintegrated rapidly such that the single pellet had fragmented into 15 to 20 pieces. This fragmentation continued and by day 30, 27 to 37 fragments were present in the vial. The 2.5% paclitaxel-loaded 30:70 (TB:MePEG350) composition fragmented more than when it was loaded with 5% paclitaxel or 10% paclitaxel; the greater the paclitaxel loading, the less fragmentation occurred. This data revealed that higher concentrations of paclitaxel in the paste prevented pellet breakdown.

### Example 11

### Dissolution of Water Soluble Polymer from the Insoluble Polymer by Weight Loss and Gel Permeation Chromatography

Weight loss and gel permeation chromatography were used to investigate whether the water soluble polymer (MePEG) dissolved out of the insoluble polymer (TB)-MePEG blend. Polymer blends of TB and MePEG in various ratios were prepared. The blends were heated to 55° C. Exact weights of the molten pastes (300 mg+/−2 mg) were poured into 15 mL Kimax test tubes. Distilled water was then added to a volume of 15 mL and the tubes were placed in a New Brunswick Scientific orbital shaker at 90 rpm at 37° C. At designated times each set of tubes (n=3) were taken from incubation, the water was removed and the polymer blends were dried in a vacuum oven at 50° C. After two days the samples were removed, reweighed and the weight loss was calculated. The molecular weights and relative compositions of the incubated paste blends were determined at ambient temperature by GPC using a Shimadzu RID-6A refractive index detector coupled to a 50A HP Pigel column with a mobile phase of chloroform at a flow rate of 1 mL/min. The injection volume was 20 uL using a polymer concentration of 0.25% (w/v). The molecular weights of the polymers were determined relative to polystyrene standards.

Results:

3.1. Weight Loss

Initial in vitro weight loss studies were carried out using 30:70 (TB:MePEG), 50:50, 70:30, and 90:10 polymer blends. A 50:50, 10% paclitaxel loaded blend was included to indicate any change in weight loss with drug loading. Pure triblock copolymer was used as a control to demonstrate no weight loss with the absence of MePEG.

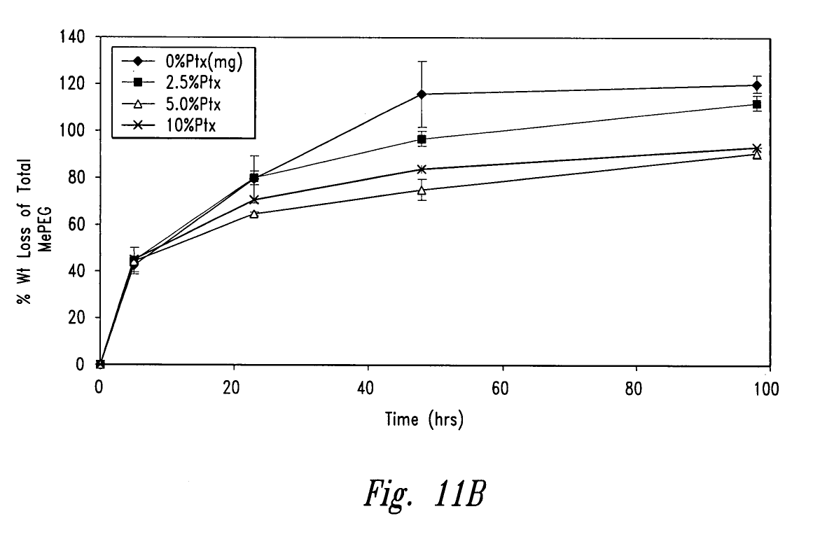

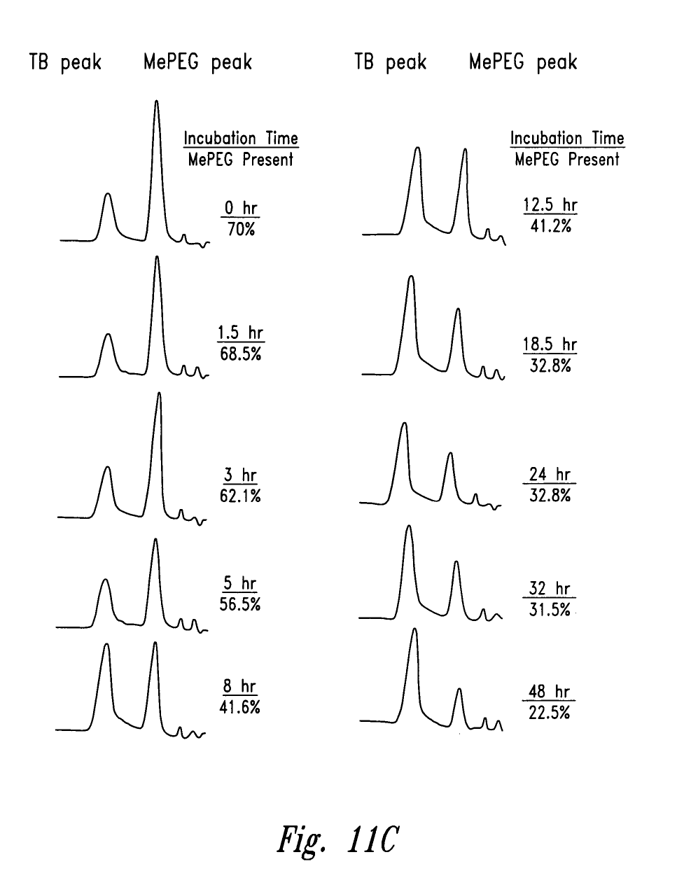

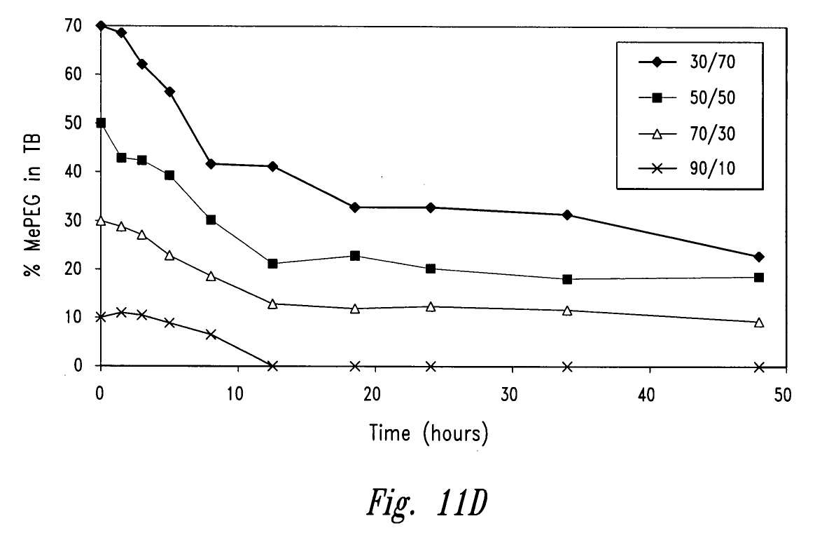

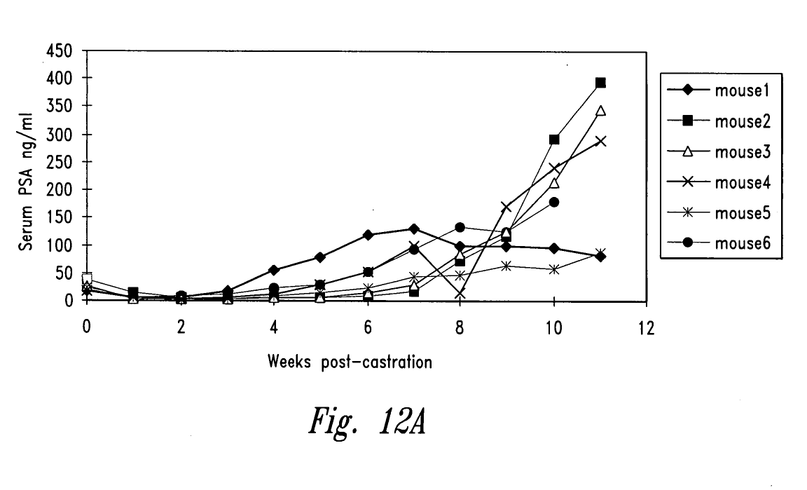

Weight loss was assumed to be a result of the dissolution of MePEG from the base triblock copolymer. Percentage values were therefore calculated as the weight change from before and after incubation divided by the total amount of MePEG present in the 300 mg polymer sample used in each test tube. FIG. 11A shows the percentage of MePEG lost from each sample at various time intervals.

The weight changes of each blend reflect a three-phase model; an initial burst phase in the first 5 hours followed by approximately 30 hours of intermediate weight loss and finishing with slow sustained weight loss. The pure TB samples showed relatively no weight change.

It has been proposed that when applied in vivo, polymer paste formulations lose their water-soluble MePEG component into the surrounding aqueous environment leaving the drug in the hydrophobic base polymer TB. On the assumption of this model, weight changes seen at various times after in vitro incubation in an aqueous medium demonstrate the progressive loss of the hydrophilic MePEG. The 30:70 blend shows the highest initial weight loss, which then slows to position itself between the 90:10 and 70:30 curves. At 96 hours the final percentage of MePEG lost was 107.9%+/−6.5%. This may be due to the lack of cohesion exhibited by the water saturated paste allowing for small particles to be lost along with the removal of the aqueous medium and consequently offsetting the weight loss profile. In addition, because of the high MePEG content, these samples are exhibiting rapid water hydration and MePEG dissolution. To clarify what effect the concentration of paclitaxel had on the loss of MePEG into the aqueous medium, another weight loss study was done using a 40:60 polymer blend loaded with 2.5, 5.0, and 10% paclitaxel, shown in FIG. 1B.

As with the formulations carrying no paclitaxel shown in FIG. 11A, these formulations show a weight loss profile of rapid, intermediate and slow sustained weight loss. At 96 hours of incubation, the percent of MePEG lost for the 0, 2.5, 5, and 10% paclitaxel loaded 40:60 blends were 120.2%+/−3.8%, 112.1%+/−3.1%, 93.0%+/−2.3%, and 93.0%+/−0.7%, respectively.

3.2. Gel Permeation Chromatography (GPC)

GPC chromatograms were obtained from one of each set of the polymer blends incubated in an aqueous environment to determine weight loss. The percent of MePEG remaining in each sample was calculated from the area under the curve representing MePEG divided by the total area under the curve for both MePEG and TB. FIG. 10C is an example of the chromatographs for the 30:70 blend.

As the time of incubation for each of the sample increased, the proportion of MePEG present in the paste blend begins to decrease. This is represented by the TB peak to the left becoming progressively larger while the MePEG peak on the right becomes comparatively smaller. The relative composition of MePEG present in the 30:70, 50:50, 70:30 and 90:10 blends used in the weight loss study are shown in FIG. 11D.

At an incubation time of 48 hours the percent of MePEG remaining in the 30:70, 50:50, 70:30, and 90:10 samples were 22.8%, 18.6%, 9.2%, and 0%, respectively. The 30:70 blend gives three phases of MePEG loss—a rapid MePEG loss up to 8 hours of incubation followed by an intermediate weight loss from 8 hours to 18.5 hours. From 18.5 hours to 48 hours there is a slow sustained loss of MePEG. The 50:50, 70:30 and 90:10 blends show a two phase MePEG loss profile. The first 12.5 hours describes intermediate weight loss, however, the blends with the higher proportion of MePEG show a steeper curve. This indicates that at the earlier stages of incubation in an aqueous environment the rate of MePEG lost from the triblock copolymer is faster with increasing concentrations of MePEG.

### Example 12

### Effect of Intratumoral Paste Injection on Serum Prostate-Specific Antigen Levels and Tumor Growth in Mice

Male 6 to 8 week old athymic nude mice (BALB/c strain) were purchased from Charles River Laboratory (Montreal, Quebec). LNCaP cells were maintained in RPMI 1640 supplemented with 5% FBS (Life Technologies Inc., Burlington, Canada).

All animals were anesthetized with methoxyfluorane before inoculation of LNCaP cells. To establish subcutaneous tumors, 1×10⁶ LNCaP cells were suspended in 75 μl of RPMI 1640 plus 5% FBS and 75 μl of Matrigel (Collaborative Biomedical Laboratories, Bedford, Mass.) and injected via 27 gauge needle into the subcutaneous space of the flank region. Tumors were measured twice weekly using calipers and their volumes were calculated by the formula L×W×H×0.5236 (Gleave et al., Cancer Res. 51:3753-3761, 1991).

Blood samples were obtained by tail vein incision of mice at specified times as previously described (Gleave et al., Cancer Res. 52:1598-1605, 1992). Serum prostate-specific antigen (PSA) levels were determined by an enzymatic immunoassay kit with a lower limit of sensitivity of 0.2 ng/mL (Abbott IMX, Montreal, Canada) according to the manufacturer's protocol.

When serum PSA levels reached 10 ng/mL, mice were anesthetized using methoxyfluorane and castrated via abdominal approach. The animals were castrated in this study, because paclitaxel might have affected androgen-regulated serum PSA production and complicated interpretation of results. Immediately following castration, mice were randomly divided into three groups termed, Control, Early Treatment and Late Treatment. Serum PSA levels were monitored in all mice twice a week.

Three weeks after castration, the Early treatment group was treated with 10% paclitaxel-loaded paste by warming the syringe containing the paste to 37° C. and injecting 100 μl of paste through a 22-gauge needle directly into the subcutaneous tumor mass. In this study, the paste was comprised of 40:60 TB:MePEG350 with paclitaxel at 10% loading (w/w). Warming the syringe was not necessary for this procedure, however, the composition was more fluid when warmed to 37° C. At the same time, control mice were treated with either the control paste (no drug) or were given no treatment. The Late treatment group was given subcutaneous intratumoral injections of 100 μl of the drug-loaded paste when the serum PSA levels began to rise (i.e., when the tumors became hormone-independent).

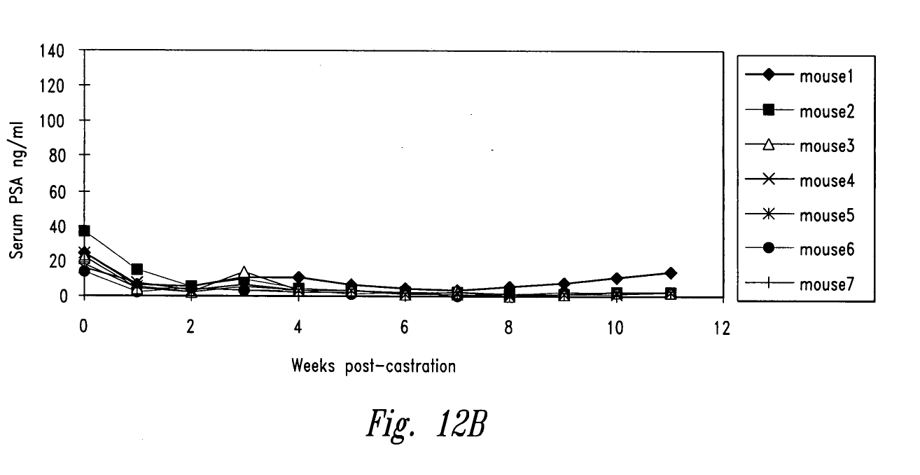

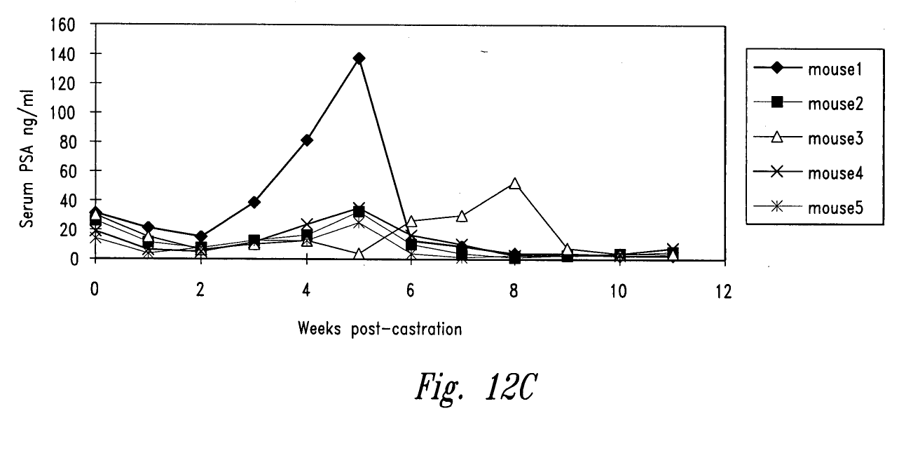

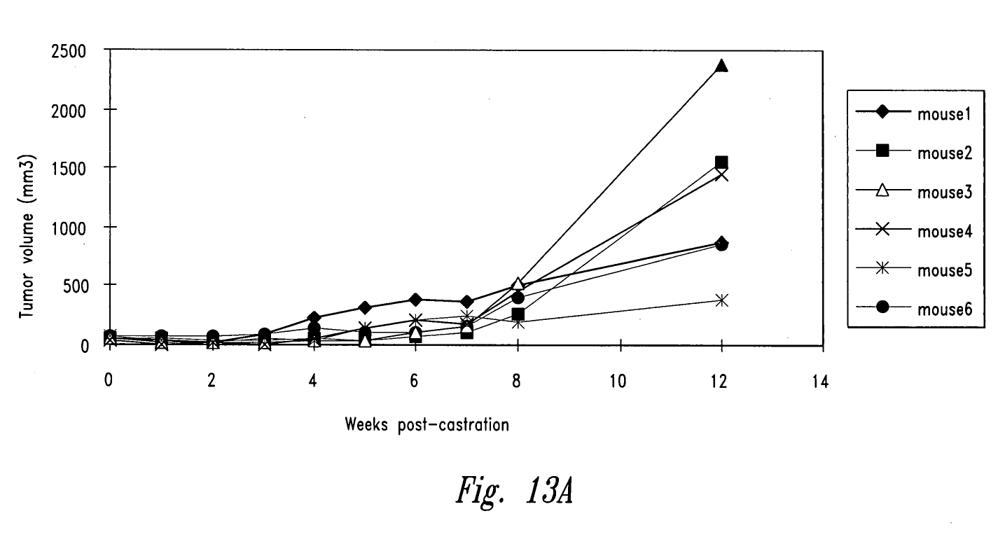

Initial pilot studies were performed to determine the biocompatibility of the polymeric paste and to determine whether the intratumoral injection of the control paste (no drug) had any effect on tumor growth rates. These experiments showed no obvious local or systemic diverse reactions to the injection of control paste when injected either intratumorally or in other subcutaneous areas. Also, the paste had no effect on tumor growth rates as compared to animals that were left untreated. The untreated control mice received no other treatment than castration. FIG. 12A shows that the control mice responded to castration by a drop in all the serum PSA levels. However, serum PSA levels increased in all animals by week 4 to 6 (post-castration). By week 10, serum PSA levels increased to levels between 50 and 300 ng/mL.

Three weeks after castration, all seven mice in the Early Treatment Group exhibited a drop in serum PSA levels from 21.9 ng/mL (mean value)±7.6 ng/mL (SD) to 7.26 ng/mL±3.8 ng/mL (FIG. 12B). At this time, all seven mice were treated with 10% paclitaxel-loaded paste by intratumoral injection of 100 μl of paste into each tumor. By week seven, all mice in this group had exhibited a decrease in serum PSA levels to 1.83 ng/mL±1.01 ng/mL. By week 11, one mouse had a slightly elevated level of 13 ng/mL, but all other serum PSA levels remained low (2.2 ng/mL±0.25 ng/mL).

Two weeks after castration, all five mice in the Late Treatment Group had reduced serum PSA levels from 24.36 ng/mL±7.6 ng/mL to 8.72 ng/mL±3.7 ng/mL (FIG. 12C). By week five, four mice had serum PSA levels that were higher than at week 0 (pre-castration) and these mice were treated with 10% paclitaxel-loaded paste. The one remaining mouse was treated at week 8 when the serum PSA level increased to 52 ng/mL. Following treatment, serum PSA levels in all mice decreased dramatically. This effect was particularly distinct for the two mice with the highest pre-treatment serum PSA levels.

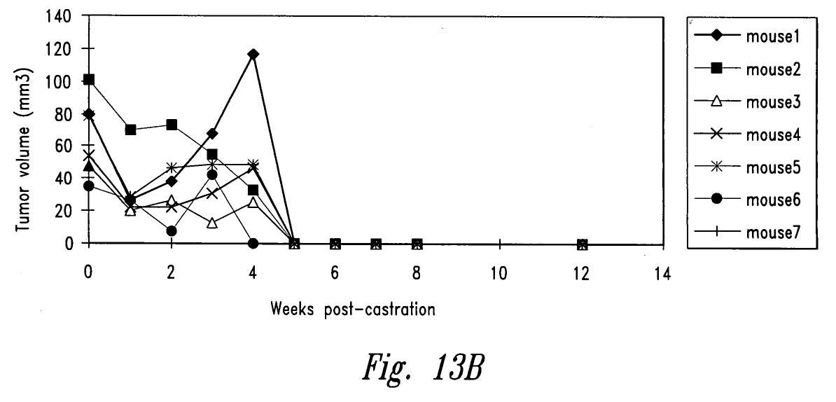

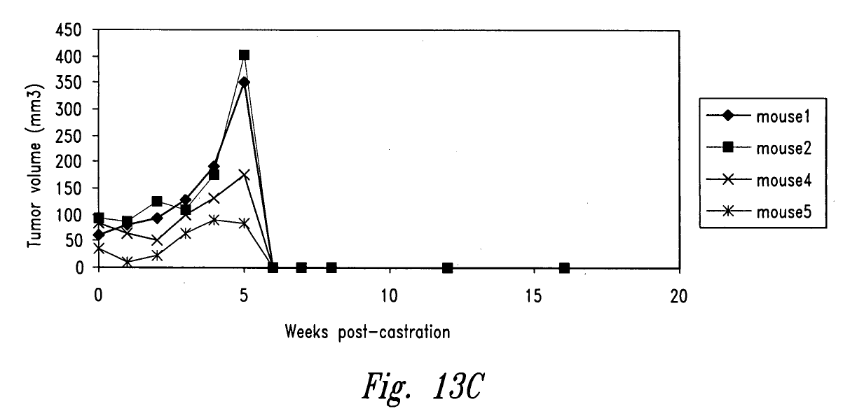

All mice treated with control paste (no drug) had rapid growth of the subcutaneous prostate tumors as shown in FIG. 13A. These growth rates matched those in untreated animals. By week ten, the mice in the untreated control group (no paste) had tumors approaching the size of 1000 mm³. At this time, the mice were sacrificed for humanitarian reasons. Following castration, there was a slight drop in tumor volume, but by week 3, all three tumors had begun to grow rapidly and growth continued until the animal was sacrificed at week 10.

In the Early Treatment Group, there was no further measurable increase in tumor volume as shown in FIG. 13B. All seven mice in this group responded identically, so that there was no detectable increase in tumor volume in any tumor in any mouse.

All five mice in the Late Treatment Group responded to paclitaxel treatment in the same manner such that tumor growth halted and tumor mass decreased (FIG. 13C). At the termination of this experiment (time of sacrifice), all treated subcutaneous areas were excised. There was no visible evidence of residual tumors in these mice.

Actively growing tumor cells were not found in paclitaxel treated mice from either group, and residual tissue from the paste implant area was determined to be composed of dead cells or cells undergoing apoptosis. Three large tumor masses were observed beneath the skin of control mice at week seven. The presence of lesions on the skin surface were observed at the treatment site of a mouse from the Late Treatment Group at week seven, indicating that the injection sites did not heal quickly. Subcutaneous masses of solidified paste at these injection sites were observed. Yet no tumor masses were seen at the sites. In some mice, the injection site became ulcerated due to the animal scratching the area. In one animal, the injection site by the front leg healed well, whereas the site by the rear leg had a scab present. By week ten, there was no evidence of scabs on any animals.

During this study, it was observed that, at 2 to 3 weeks post-castration (Early), the tumors remained androgen-dependent whereas at 3 to 5 weeks (Late) untreated LNCaP tumors became androgen independent as evidenced by increasing serum PSA levels and tumor volumes. The Early Treatment Group provided a model for localized tumors in humans, which is also androgen dependent. Although the paclitaxel paste formulation was designed for the treatment of localized, androgen dependent, non-metastatic prostate tumors, it is important that the treatment was effective against both androgen dependent and independent tumors since both of these tumor types might ultimately become the therapeutic target for paclitaxel.

The paclitaxel-loaded formulation used in this study was found to solidify to a solid form in aqueous media at 37° C. within 1 hour and to release paclitaxel quickly as shown in FIG. 9C. In vivo, this paste was applied easily through a 22-gauge needle and set to a semi-solid implant in approximately 1 to 2 hours. This formulation was determined to be efficacious against subcutaneous LNCaP tumors in the 12 mice that made up the two treatment groups (Early and Late).

Serum PSA levels in this subcutaneous LNCaP model have been shown to be directly related to tumor volume and these levels dropped rapidly and remained low (below 15 ng/mL) following treatment whereas levels in control mice increased up to 400 ng/mL. Similarly, there was no detectable increase in tumor size in any tumor in any animal and no growing tumor tissue could be found at post-mortem in these animals. These results clearly show the efficacy of this treatment regimen.

No systemic toxicity, such as weight loss, listlessness or gait disturbance, characteristic of systemic paclitaxel, was observed in any animal during treatment. However, local ulceration was present in some mice, probably due to irritation and scratching of the skin followed by paclitaxel inhibition of wound healing at the irritation/scratch site, as has been previously reported for this drug.

The use of subcutaneous injections of LNCaP cells in mice provides an ideal model for the in vivo study of the effects of chemotherapeutic agents against prostate cancer (Carter et al., Prostate, 16:39-48, 1990; Gleave et al., Cancer Res., 52:1598-1605, 1992; Sato et al., Cancer Res., 57:1584-1589, 1997). Tumors grown from LNCaP cells are non-metastatic and secrete PSA, which correlates with tumor volume. Therefore, assessment of tumor growth is easily determined. In addition, the subcutaneous location of this tumor allows for simple and serial measurement of tumor volume (Gleave, et al., Cancer Res., 52:1598-1605, 1992).

### Example 13

### The Use of Paclitaxel Loaded Pastes for the Perivascular Treatment of Restenosis in Rats

Balloon injury in the rat carotid artery (paste injection). Wistar rats weighing 400 g to 500 g are anesthetized with halothane (5% induction −1.5% maintenance). A vertical incision is made over the trachea and the left external carotid artery is exposed. Connective tissue around the left common carotid artery is left untouched. Two ligatures are placed around the external carotid artery and an arteriotomy is made between them. A 2 French Fogarty balloon is introduced into the external carotid artery and pushed into the left common carotid artery and the balloon is inflated with saline. The balloon is passed up and down the entire length of the carotid artery three times to denude the endothelium. The balloon is removed and the ligatures tied off on the external carotid artery. Paclitaxel in a polymeric paste or the carrier paste alone is injected through a 24 G angiocatheter between a distal segment (1 cm long) of the common carotid artery and the surrounding connective tissue. Typically, 0.1 to 0.2 mL of paste is injected. The wound is then closed. After 14 or 28 days, the rats are sacrificed and pressure perfused at 100 mmHg with 10% buffered formaldehyde. Both carotid arteries are harvested and processed for histology. Serial cross-sections are cut every 2 mm within and outside the treated segment of the injured left common carotid artery and at corresponding levels in the control right carotid artery. Sections are stained with hematoxylin-and-eosin and Movat's stains. Morphometric analysis is performed to quantify luminal narrowing and intimal hyperplasia. Medial cell count is carried out to assess toxicity.

Mice were treated with either 40:60 or 70:30 (TB:MePEG) blends containing either none, 1%, 5% or 10% paclitaxel. Both 40:60 and 70:30 blends were easy to inject through the 24 gauge needle. Control pastes (no paclitaxel) caused no inhibition of restenosis or toxic side effects. The pastes did not cause any seroma, necrosis, skin irritation or inflammation at the site of implantation and did not cause any cell loss in the arterial wall. These data indicate excellent biocompatibility of the paste in rats at the carotid artery site. Rats treated with 10% paclitaxel loaded 40:60 paste showed complete inhibition of intimal hyperplasia, i.e., complete inhibition of restenosis. However, the use of 10% loaded paste caused some seroma and necrosis at the site of implantation and some cell loss in the medial and adventitial layers of arterial wall indicating some toxicity. However a 1% paclitaxel loaded 40:60 paste produced an approx. 50% inhibition of restenosis in the carotid artery with no signs of gross toxicity at the site of implantation.

These results show that a therapeutic window exists for the paclitaxel loaded 40:60 paste since 1% loaded paste gives 50% inhibition of restenosis with no toxicity whereas 10% loaded gives full inhibition but some toxic side effects. The 5% paclitaxel loaded 70:30 paste gave full efficacy in the treatment of carotid artery restenosis in rats (n=4) and 50% inhibition in 1 rat. This inhibition of restenosis was associated with some degree of toxicity at the site of implantation and some cell loss in the arterial wall. However the toxicity seen with all these paclitaxel loaded paste formulations was not severe indicating that these pastes offer an acceptable and effective non invasive perivascular treatment for restenosis.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

1. A polymeric drug delivery system, comprising:
(a) a biodegradable water insoluble polymer that is a solid or wax at 37° C.;
(b) a biodegradable water soluble polymer that is a liquid at 25° C.; and
(c) a hydrophobic drug, wherein said polymeric drug delivery system is a liquid or paste at 25° C.

2. The polymeric drug delivery system of claim 1 wherein said water insoluble polymer is a polymer selected from the group consisting of polylactic acid, polyglycolic acid, polycaprolactone, polyanhydride, polybutyric acid, polyacrylic acid, and polymethacrylate.

3. The polymeric drug delivery system of claim 1 wherein said water insoluble polymer is a block copolymer, said block copolymer comprising a block selected from the group consisting of polylactic acid, polyglycolic acid, polycaprolactone, polyanhydride, polybutyric acid, polyacrylic acid, and polymethacrylate.

4. The polymeric drug delivery system of claim 3 wherein said block copolymer comprises a hydrophilic block selected from the group consisting of polyalkylene oxide and polysaccharide.

5. The polymeric drug delivery system of claim 3 wherein said water insoluble polymer is a triblock copolymer having the formula ABA, wherein each A is a hydrophobic block, and wherein B is a hydrophilic block.

6. The polymeric drug delivery system of claim 5 wherein said hydrophobic block is a polyester.

7. The polymeric drug delivery system of claim 6 wherein said polyester is a poly(α-hydroxy acid).

8. The polymeric drug delivery system of claim 7 wherein said poly(α-hydroxy acid) is poly(glycolic acid) or poly(lactic acid).

9. The polymeric drug delivery system of claim 6 wherein said hydrophilic block is a polyalkylene oxide.

10. The polymeric drug delivery system of claim 9 wherein said polyalkylene oxide is polyethylene glycol.

11. The polymeric drug delivery system of claim 9 wherein said polyester and said polyalkylene oxide components of said triblock copolymer are linked by caprolactone links.

12. The polymeric drug delivery system of claim 11 wherein said triblock copolymer comprises [poly(DL-lactide-co-ε-caprolactone)]-[polyethylene glycol]-[poly(DL-lactide-co-ε-caprolactone)].

13. The polymeric drug delivery system of claim 1 wherein said water soluble polymer is polyethylene glycol or methoxypolyethylene glycol.

14. The polymeric drug delivery system of claim 12 wherein said water soluble polymer is methoxypolyethylene glycol having a number average molecular weight of about 100-500.

15. The polymeric drug delivery system of claim 14 wherein said triblock copolymer (TB) and said methoxypolyethylene glycol (MePEG) are present in said polymeric drug delivery system at a weight ratio of TB:MePEG within the range of 30:70 to 90:10.

16. The polymeric drug delivery system of claim 1 wherein said water insoluble polymer is a triblock copolymer of the formula ABA, wherein A is a block of residues comprising residues which remain after polymerization of one or more monomers selected from the group consisting of hydroxyacetic acid, 2-hydroxypropionic acid and 6-hydroxyhexanoic acid, B is a block of residues comprising residues which remain after polymerization of one or more monomers selected from the group consisting of alkylene oxide and alkylene glycol, and the triblock copolymer is a liquid at a temperature within the range of 25-40° C.

17. The polymeric drug delivery system of claim 1 wherein said water insoluble polymer is a triblock copolymer of the formula ABA, wherein A is a block of residues comprising residues which remain after polymerization of one or more monomers selected from the group consisting of hydroxyacetic acid, 2-hydroxypropionic acid and 6-hydroxyhexanoic acid, B is a block of residues comprising residues which remain after the polymerization of one or more monomers selected from the group consisting of alkylene oxide and alkylene glycol, and the copolymer is a paste at a temperature within the range of 25-40° C.

18. The polymeric drug delivery system of claim 1 wherein said water insoluble polymer is a triblock copolymer of the formula ABA, wherein A is a block of residues comprising residues which remain after polymerization of one or more monomers selected from the group consisting of hydroxyacetic acid, 2-hydroxypropionic acid and 6-hydroxyhexanoic acid, B is a block of residues comprising residues which remain after the polymerization of one or more monomers selected from the group consisting of alkylene oxide and alkylene glycol, and the copolymer is not a solid at 25° C.

19. The polymeric drug delivery system of claim 1 wherein the weight of said hydrophobic drug represents a percentage of the total weight of said polymeric drug delivery system within the range of 2-30%.

20. The polymeric drug delivery system of claim 1 wherein said hydrophobic drug is selected from the group consisting of amphotericin, anthralin, beclomethasone, betamethasone, camptothecin, curcumin, dexamethasone, indomethacin, genistein, lidocaine, insulin, nystatin, paclitaxel, tetracycline, tretinoin, cromoglycate, levobunolol, and terbinafine.

21. The polymeric drug delivery system of claim 20 wherein said hydrophobic drug is selected from the group consisting of paclitaxel, camptothecin, amphoterecin, nystatin, tretinoin, genistein, and curcumin.

22. The polymeric drug delivery system of claim 20 wherein said hydrophobic drug is paclitaxel.

23. The polymeric drug delivery system of claim 1, comprising at least two drugs.

24. A method for delivering a drug to a subject, comprising the administration of a polymeric drug delivery system that comprises (a) a biodegradable water insoluble polymer that is a solid or wax at 37° C., (b) a biodegradable water soluble polymer that is a liquid at 25° C., and (c) a hydrophobic drug, wherein said polymeric drug delivery system is a liquid or paste at 25° C.

25. The method of claim 24 wherein said polymeric drug delivery system is administered to said subject by a method selected from the group consisting of intraperitoneal injection, intraarticular injection, intraocular injection, intratumoral injection, perivascular injection, subcutaneous injection, intracranial injection, and intramuscular injection.

26. The method of claim 24 wherein said polymeric drug delivery system is administered to said subject by application on a surgically exposed tissue.

27. The method of claim 24 wherein said polymeric drug delivery system is administered to said subject by a mode selected from the group consisting of periophthalmic application, administration inside the eyelid, intraoral administration, intranasal administration, intrabladder administration, intravaginal administration, intraurethral administration, intrarectal administration, and application to the adventitia of an internal organ.

28. The method of claim 24 wherein said subject is a mammal.

29. The method of claim 28 wherein said mammal is a human.

30. The method of claim 28 wherein said mammal is a farm or domestic animal.

31. A method of preparing a polymeric drug delivery system, comprising the blending of: (a) a biodegradable water insoluble polymer that is a solid or wax at 37° C., (b) a biodegradable water soluble polymer that is a liquid at 25° C., and (c) a hydrophobic drug, wherein said polymeric drug delivery system is a liquid or paste at 25° C.

32. The method of claim 31 wherein said hydrophobic drug is not mixed with an organic solvent prior to said blending step.

33. A triblock copolymer of the formula ABA, wherein A is a block of residues comprising residues which remain after polymerization of one or more monomers selected from the group consisting of hydroxyacetic acid, 2-hydroxypropionic acid and 6-hydroxyhexanoic acid, B is a block of residues comprising residues which remain after the polymerization of one or more monomers selected from the group consisting of alkylene oxide and alkylene glycol, and the copolymer has a consistency, at a temperature within the range of 25-40° C., selected from the group consisting of a paste and a liquid, or has a non-solid consistency at 25° C.

34. The copolymer of claim 33 wherein block A consists essentially of residues having the structure resulting from the polymerization of monomers selected from the group hydroxyacetic acid, 2-hydroxypropionic acid and 6-hydroxyhexanoic acid.

35. The copolymer of claim 33 wherein block A comprises residues having the structure resulting from the polymerization of 2-hydroxypropionic acid.

36. The copolymer of claim 33 wherein block A consists essentially of residues having the structure resulting from the polymerization of 2-hydroxypropionic acid.

37. The copolymer of claim 33 wherein block A comprises residues having the structure resulting from the polymerization of 6-hydroxyhexanoic acid.

38. The copolymer of claim 33 wherein block A comprises residues having the structure resulting from the polymerization of 2-hydroxypropionic acid and 6-hydroxyhexanoic acid.

39. The copolymer of claim 33 wherein block A consists essentially of residues having the structure resulting from the polymerization of 2-hydroxypropionic acid and 6-hydroxyhexanoic acid.

40. The copolymer of claim 33 wherein block A contains residues having the structure resulting from the polymerization of 2-hydroxypropionic acid and 6-hydroxyhexanoic acid in a 2-hydroxypropionic acid:6-hydroxyhexanoic acid weight ratio of 40-60:60-40.

41. The copolymer of claim 33 wherein the A block is a random copolymer.

42. The copolymer of claim 33 wherein block B comprises residues having the structure resulting from the polymerization of ethylene oxide.

43. The copolymer of claim 33 wherein block B is a CDC triblock copolymer wherein C and D are selected from homopolymers of ethylene oxide and propylene oxide.

44. The copolymer of claim 33 wherein block B has a number average molecular weight of less than or equal to 8,000.

45. The copolymer of claim 44 wherein the molecular weight is less than or equal to 1,000 and at least 100.

46. The copolymer of claim 33 wherein the B block provides 10-50% of the weight of the copolymer.

47. The copolymer of claim 33 wherein at least 50% of the copolymer is biodegradable.

48. A drug delivery system comprising a drug in combination with a triblock copolymer of the formula ABA, wherein A is a block of residues comprising residues which remain after polymerization of one or more monomers selected from the group consisting of hydroxyacetic acid, 2-hydroxypropionic acid and 6-hydroxyhexanoic acid, B is a block of residues comprising residues which remain after the polymerization of one or more monomers selected from the group consisting of alkylene oxide and alkylene glycol, and the copolymer has a consistency, at a temperature within the range of 25-40° C., selected from the group consisting of a paste and a liquid, or has a non-solid consistency at 25° C.

49. The drug delivery system of claim 48 wherein the drug is selected from a peptide, protein, antigen, vaccine, anti-infective, antibiotic, antimicrobial, antiallergenic, steroid, decongestant, miotic, anticholinergic, sympathomimetic, sedative, hypnotic, psychic energizer, tranquilizer, analgesic, antimalarial and antihistamine.

50. The drug delivery system of claim 48 wherein the drug is paclitaxel.

51. The drug delivery system of claim 48 wherein the drug provides 0.1% to 10% of the total weight of the system.

52. A method of administering a drug to a subject comprising contacting the subject with a drug delivery system comprising a drug in combination with a triblock copolymer of the formula ABA, wherein A is a block of residues comprising residues which remain after polymerization of one or more monomers selected from the group consisting of hydroxyacetic acid, 2-hydroxypropionic acid and 6-hydroxyhexanoic acid, B is a block of residues comprising residues which remain after the polymerization of one or more monomers selected from the group consisting of alkylene oxide and alkylene glycol, and the copolymer has a consistency, at a temperature within the range of 25-40° C., selected from the group consisting of a paste and a liquid, or has a non-solid consistency at 25° C.

53. The method of claim 52 wherein the drug delivery system is injected directly into a solid tumor of the subject.

54. The method of claim 52 wherein the drug delivery system is applied to a tumor resection cavity.

55. The method of claim 52 wherein the tumor resection cavity contains cancer cells.

56. The method of claim 52 wherein the drug kills cancer cells.

57. The method of claim 52 wherein the drug delivery system is topically applied to tissue of the subject.

58. The method of claim 52 wherein the drug prevents post-surgical adhesion.

59. The method of claim 52 wherein the drug delivery system is applied perivascularly to the subject.

60. The method of claim 52 wherein the drug treats restenosis.

61. The method of claim 52 wherein the drug delivery system is injected intra-articularly to the subject.

62. The method of claim 52 wherein the drug treats arthritis.
